(19) United States
(12) Patent Application Publication
(10) Pub. No.: US 2012/0009601 A1
(43) Pub. Date: Jan. 12, 2012

(54) METHODS AND COMPOSITIONS FOR REPROGRAMMING CELLS

(75) Inventors: Hisashi Moriguchi, Chiba (JP); Raymond T. Chung, Chestnut Hill, MA (US)

(73) Assignee: THE GENERAL HOSPITAL CORPORATION, Boston, MA (US)

(21) Appl. No.: 13/177,662

(22) Filed: Jul. 7, 2011

Related U.S. Application Data
(60) Provisional application No. 61/362,538, filed on Jul. 8, 2010.

(51) Int. Cl. G01N 33/566; C12N 5/071

(52) U.S. Cl. 435/7.21; 435/366

(57) ABSTRACT

The present invention is directed, in part, to methods and compositions comprising inhibitors of micro RNA-145 and activators of the TGF-β signaling pathway to permit reprogramming using only small molecule compounds. Also described herein are methods to distinguish cancer cells or cells having cancerous potential in human iPS cell populations, based on determining the balance of p21-p53 expression levels, or ratio thereof in reprogrammed cells. In further aspects, methods and compositions to cause redifferentiation of a hepatoma cell to a hepatocye-like cell using acyclic retinoid and inhibitors of AKR1B10 are provided.

### CROSS-REFERENCE TO RELATED APPLICATIONS

This application claims benefit under 35 U.S.C. §119(e) of U.S. Provisional Patent Application Ser. No. 61/362,538 filed on 8 Jul. 2010, the contents of which are incorporated herein by reference in their entirety.

### FIELD OF THE INVENTION

The invention relates to methods and compositions for reprogramming cells, and evaluating cancerous potential of reprogrammed cells.

### BACKGROUND

Embryonic stem cells (ES cells) were first derived in 1981 from the inner cell mass (ICM) of murine pre-implantation blastocyst embryos (Evans and Kaufman, 1981; Martin, 1981). ES cells are pluripotent and demonstrate the ability to expand indefinitely in vitro while retaining the capacity to generate ectoderm-, endoderm-, and mesoderm derivatives both in vitro and in vivo. The discovery of murine ES cells was a major breakthrough in developmental biology, since it allowed the study of mammalian gene function in vivo, through the application of transgenic and knockout technologies. The subsequent derivation of human ES cells was another breakthrough for insights into human development and disease.

Mammalian development involves a progressive restriction of developmental potential as the totipotent zygote transits through the pluripotent inner cell mass (ICM) and eventually gives rise to a multitude of differentiated cell types that generally lack the ability to revert back to a less specialized state. Embryonic stem cells (ESCs) derived from the ICM of blastocyst stage embryos can be propagated in vitro, and these cells retain pluripotency, which is the capability to give rise to all cell types of the embryo proper (Rossant, 2008). These features of ESCs have made them an attractive tool for early developmental studies as well as a resource for potential applications in regenerative medicine (B. Feng et al., Cell Stem Cell 2009, 4:301-312).

Recent work has implicated a variety of transcription factors and epigenetic modifiers, as well as agents and methods for targeting several cell signaling pathways, as useful in methods for reprogramming cells into a pluripotent state. Some methods have described that inhibition of the TGF-β/Activin/Nodal signaling pathway has positive effects, i.e., promotes, on cellular reprogramming, (Yu and Thomson, 2008; Li et al., 2009).

Besides transcription factors, epigenetic modifiers, and agents and methods for targeting cell signaling pathways, microRNAs have been postulated as playing an important role in reprogramming, commensurate with their emerging role in the maintenance of ES cells (Ivey et al., 2008). To date, however, only one particular microRNA, mir-302, which is expressed abundantly in human ES cells, has been implicated in reprogramming (Lin et al., 2008).

### SUMMARY OF THE INVENTION

The methods and compositions described herein are based, in part, on the novel discovery that inhibitors of micro RNA-145 and activators of the TGF-β signaling pathway permit reprogramming using only small molecule compounds of human somatic cells, such as human somatic cells having some endogenous expression of Oct 3/4 and Sox2. Such cells reprogrammed using the compositions and methods described herein are referred to as “chemically induced pluripotent stem” (“ChiPS”) cells.

In other aspects, described herein are methods to distinguish cancer cells or cells having cancerous potential in human iPS cell populations, generated using any method. These aspects are based, in part, on the discovery that the balance of p21-p53 expression levels, or ratio thereof, is important in order to distinguish or identify cancerous cells or cells with cancerous potential derived from human iPS cells. Further, it was found that PRIMA-1 can be used to avoid the malignant transformations of human iPS cells that carry mutant p53, and allows for exclusion of iPS cells having malignant potential, and permits selection and/or isolation of the safest human iPS cells.

In further aspects, methods and compositions are provided that are based, in part, on the novel discovery that acyclic retinoid and inhibitors of AKR1B10 can be used to cause redifferentiation of a hepatoma cell to a hepatocye-like cell.

Accordingly, in one aspect a cell-growth composition comprising an inhibitor of microRNA-145 and an activator of TGF-β signaling is provided. In another aspect, a cell-growth composition consisting essentially of an inhibitor of microRNA-145 and an activator of TGF-β signaling is provided.

In some embodiments of these aspects and all such aspects described herein, an inhibitor of microRNA-145 is a small molecule or a nucleic acid inhibitor of microRNA-145. In some embodiments, the nucleic acid inhibitor of microRNA-145 is a anti-microRNA-145 LNA oligonucleotide or a 2′OMe-microRNA-145. In some embodiments, the activator of TGF-β signaling is a TGF-β polypeptide or an active fragment thereof, a fusion protein comprising a TGF-β polypeptide or an active fragment thereof, an agonist antibody to a TGF-β receptor, a small molecule agonist of a TGF-β receptor, or a TGF-β production stimulator. In some embodiments, the activator of TGF-β signaling is a recombinant TGF-β polypeptide or an active fragment thereof. In some embodiments, the inhibitor of microRNA-145 and the activator of TGF-β signaling comprise 2′ OMe-microRNA-145 and recombinant TGF-β polypeptide or an active fragment thereof, respectively.

In another aspect, a composition comprising a human somatic cell or population of human somatic cells in admixture with an inhibitor of microRNA-145 and an activator of TGF-β signaling is provided. In one aspect, a composition consisting essentially of a human somatic cell or population of human somatic cells in admixture with an inhibitor of microRNA-145 and an activator of TGF-β signaling is provided.

In some embodiments of these aspects and all such aspects described herein, the human somatic cell or population of human somatic cells has endogenous expression of OCT3/4. In some embodiments of these aspects and all such aspects described herein, the human somatic cell or population of human somatic cells has endogenous expression of SOX2. In some embodiments of these aspects and all such aspects described herein, the human somatic cell or population of human somatic cells has endogenous expression of OCT3/4 and SOX2.

In some embodiments of these aspects and all such aspects described herein, an inhibitor of microRNA-145 is a small molecule or a nucleic acid inhibitor of microRNA-145. In some embodiments, the nucleic acid inhibitor of microRNA-145 is a anti-microRNA-145 LNA oligonucleotide or a 2′OMe-microRNA-145. In some embodiments, the activator of TGF-β signaling is a TGF-β polypeptide or an active fragment thereof, a fusion protein comprising a TGF-β polypeptide or an active fragment thereof, an agonist antibody to a TGF-β receptor, a small molecule agonist of a TGF-β receptor, or a TGF-β production stimulator. In some embodiments, the activator of TGF-β signaling is a recombinant TGF-β polypeptide or an active fragment thereof. In some embodiments, the inhibitor of microRNA-145 and the activator of TGF-β signaling comprise 2′ OMe-microRNA-145 and recombinant TGF-β polypeptide or an active fragment thereof.

In one aspect, a method of generating a chemically induced pluripotent stem (ChiPS) cell from a human somatic cell is provided, the method comprising contacting a human somatic cell with an inhibitor of microRNA-145 and an activator of TGF-β signaling, thereby generating a chemically induced induced pluripotent stem cell.

In some embodiments of the aspect and all such aspects described herein, the human somatic cell or population of human somatic cells has endogenous expression of OCT3/4. In some embodiments of the aspect and all such aspects described herein, the human somatic cell or population of human somatic cells has endogenous expression of SOX2. In some embodiments of the aspect and all such aspects described herein the human somatic cell or population of human somatic cells has endogenous expression of OCT3/4 and SOX2.

In some embodiments of the aspect and all such aspects described herein, an inhibitor of microRNA-145 is a small molecule or a nucleic acid inhibitor of microRNA-145. In some embodiments, the nucleic acid inhibitor of microRNA-145 is a anti-microRNA-145 LNA oligonucleotide or a 2′OMe-microRNA-145. In some embodiments of the aspect and all such aspects described herein, the activator of TGF-β signaling is a TGF-β polypeptide or an active fragment thereof, a fusion protein comprising a TGF-β polypeptide or an active fragment thereof, an agonist antibody to a TGF-β receptor, a small molecule agonist of a TGF-β receptor, or a TGF-β production stimulator. In some such embodiments, the activator of TGF-β signaling is a recombinant TGF-β polypeptide or an active fragment thereof. In some embodiments, the inhibitor of microRNA-145 and the activator of TGF-β signaling comprise 2′OMe-microRNA-145 and recombinant TGF-β polypeptide or an active fragment thereof.

In another aspect, a kit for generating a chemically induced pluripotent stem (ChiPS) cell from a human somatic cell is provided, the kit comprising: an inhibitor of microRNA-145, an activator of TGF-β signaling, and instructions and packaging thereof.

In some embodiments of the aspect, an inhibitor of microRNA-145 is a small molecule or a nucleic acid inhibitor of microRNA-145. In some embodiments, the nucleic acid inhibitor of microRNA-145 is a anti-microRNA-145 LNA oligonucleotide or a 2′OMe-microRNA-145. In some embodiments, the activator of TGF-β signaling is a TGF-β polypeptide or an active fragment thereof, a fusion protein comprising a TGF-β polypeptide or an active fragment thereof, an agonist antibody to a TGF-β receptor, a small molecule agonist of a TGF-β receptor, or a TGF-β production stimulator. In some embodiments, the activator of TGF-β signaling is a recombinant TGF-β polypeptide or an active fragment thereof. In some embodiments, the inhibitor of microRNA-145 and the activator of TGF-β signaling comprise 2′OMe-microRNA-145 and recombinant TGF-β polypeptide or an active fragment thereof.

In another aspect, a method of assessing the risk of cancerous transformation of an induced pluripotent stem (iPS) cell is provided. In such methods, the induced pluripotent stem (iPS) cell can be generated using any method known to one of skill in the art, as well as using any of the compositions or methods described herein. The method comprises measuring an expression of p21 and measuring an expression of p53 in an induced pluripotent stem cell, and determining the ratio of expression of p21 to p53 in the induced pluripotent stem cell, wherein an increased expression of p21 relative to the expression of p53 in the induced pluripotent stem cell is indicative of a decreased risk of cancerous transformation.

In one aspect, a method of decreasing the risk of cancerous transformation of an induced pluripotent stem (iPS) cell is provided, the method comprising contacting an induced pluripotent stem cell with one or more agents that induce p21 expression in the induced pluripotent stem cell, wherein the induction of p21 expression in the induced pluripotent stem cell decreases the risk of cancerous transformation.

In some embodiments, the agent that induces p21 expression is PRIMA-1 (2,2-Bis(hydroxymethyl)-1-azabicyclo[2.2.2]octan-3-one). In some embodiments of the aspect, the agent that induces p21 expression can be selected from the group comprising PRIMA-1 (2,2-Bis(hydroxymethyl)-1-azabicyclo[2.2.2]octan-3-one); arsenic trioxide (As2O3); Daunomycin; Apicidin; 5-phenyl-2,4-pentadienoyl hydroxamic acid, N-methyl-5-phenyl-2,4-pentadienoyl hydroxamic acid, 3-methyl-5-phenyl-2,4-pentadienoyl hydroxamic acid, 4-methyl-5-phenyl-2,4-pentadienoyl hydroxamic acid, 4-chloro-5-phenyl-2,4-pentadienoyl hydroxamic acid, 5-(4-dimethylaminophenyl)-2,4-pentadienoyl hydroxamic acid, 5-phenyl-2-en-4-yn-pentanoyl hydroxamic acid, N-methyl-6-phenyl-3,5-hexadienoyl hydroxamic acid, potassium 2-oxo-6-phenyl-3,5-hexadienoate, potassium 2-oxo-8-phenyl-3,5,7-octatrienoate, 7-phenyl-2,4,6-hepta-trienoylhydroxamic acid, or derivatives thereof; and cis-Diamminedichloroplatinum. In some embodiments, the agent that induces p21 expression is any agent or compound that inhibits microRNA-106(b) family members.

In another aspect, a cell-growth composition comprising acyclic retinoid and an aldo-keto reductase family 1 B10 (AKR1B10) inhibitor is provided. In another aspect, a cell-growth composition consisting essentially of acyclic retinoid and an aldo-keto reductase family 1 B10 (AKR1B10) inhibitor is provided.

In some embodiments of these aspects, the acyclic retinoid is NIK-333 [(2E,4E,6E,10E)-3,7,11,15-tetramethyl 2,4,6,10,14-hexadecapentaenoic acid]. In some embodiments of these aspects, the AKR1B10 inhibitor is tolrestat. In some embodiments, the AKR1B10 inhibitor is epalrestat or 2-[(5Z)-5-[(E)-3-cyclohexyl-2-methylprop-2-enylidene]-4-oxo-2-thioxo-3-thiazolidinyl]acetic acid acid. In some embodiments of the aspects, the AKR1B10 inhibitor is selected from the group comprising: epalrestat or 2-[(5Z)-5-[(E)-3-cyclohexyl-2-methylprop-2-enylidene]-4-oxo-2-thioxo-3-thiazolidinyl]acetic acid or a derivative thereof; 9-methyl-2,3,7-trihydroxy-6-fluorone or a derivative thereof; or 5-O-Dicaffeoyl-epiquinic acid (3,5-DCQA) or a derivative thereof. In one embodiment, the acyclic retinoid is NIK-333 [(2E,4E,6E,10E)-3,7,11,15-tetramethyl 2,4,6,10,14-hexadecapentaenoic acid] and the AKR1B10 inhibitor is tolrestat. In one embodiment, the acyclic retinoid is NIK-333 [(2E,4E,6E,10E)-3,7,11,15-tetramethyl 2,4,6,10,14-hexadecapentaenoic acid] and the AKR1B10 inhibitor is epalrestat or 2-(5Z)-5-[(E)-3-cyclohexyl-2-methylprop-2-enylidene]-4-oxo-2-thioxo-3-thiazolidinyflacetic acid.

In another aspect, a pharmaceutical composition comprising an acyclic retinoid, an aldo-keto reductase family 1 B10 (AKR1B10) inhibitor, and a pharmaceutically acceptable carrier is provided. In one aspect, a pharmaceutical composition consisting essentially of an acyclic retinoid, an aldo-keto reductase family 1 B10 (AKR1B10) inhibitor, and a pharmaceutically acceptable carrier is provided.

In some embodiments of these aspects, the acyclic retinoid is NIK-333 [(2E,4E,6E,10E)-3,7,11,15-tetramethyl 2,4,6,10,14-hexadecapentaenoic acid]. In some embodiments of these aspects, the AKR1B10 inhibitor is tolrestat. In some embodiments, the AKR1B10 inhibitor is epalrestat or 2-[(5Z)-5-[(E)-3-cyclohexyl-2-methylprop-2-enylidene]-4-oxo-2-thioxo-3-thiazolidinyl]acetic acid. In some embodiments of the aspects, the AKR1B10 inhibitor is selected from the group comprising: epalrestat or 2-[(5Z)-5-[(E)-3-cyclohexyl-2-methylprop-2-enylidene]-4-oxo-2-thioxo-3-thiazolidinyl]acetic acid or a derivative thereof; 9-methyl-2,3,7-trihydroxy-6-fluorone or a derivative thereof; or 5-O-Dicaffeoyl-epiquinic acid (3,5-DCQA) or a derivative thereof. In one embodiment, the acyclic retinoid is NIK-333 [(2E,4E,6E,10E)-3,7,11,15-tetramethyl 2,4,6,10,14-hexadecapentaenoic acid] and the AKR1B10 inhibitor is tolrestat. In one embodiment, the acyclic retinoid is NIK-333 [(2E,4E,6E,10E)-3,7,11,15-tetramethyl 2,4,6,10,14-hexadecapentaenoic acid] and the AKR1B10 inhibitor is epalrestat or 2-[(5Z)-5-[(E)-3-cyclohexyl-2-methylprop-2-enylidene]-4-oxo-2-thioxo-3-thiazolidinyl]acetic acid.

In another aspect, a method of generating a hepatocyte-like cell from a hepatoma cell is provided, the method comprising contacting a hepatoma cell with acyclic retinoid and an aldo-keto reductase family 1 B10 (AKR1B10) inhibitor, thereby generating a hepatocyte-like cell.

In some embodiments of the aspect, the human hepatoma cell is positive for CD133.

In some embodiments of the aspect, the acyclic retinoid is NIK-333 [(2E,4E,6E,10E)-3,7,11,15-tetramethyl 2,4,6,10,14-hexadecapentaenoic acid]. In some embodiments of these aspects, the AKR1B10 inhibitor is tolrestat. In some embodiments, the AKR1B10 inhibitor is epalrestat or 2-[(5Z)-5-[(E)-3-cyclohexyl-2-methylprop-2-enylidene]-4-oxo-2-thioxo-3-thiazolidinyl]acetic acid. In some embodiments of the aspects, the AKR1B10 inhibitor is selected from the group comprising: epalrestat or 2-[(5Z)-5-[(E)-3-cyclohexyl-2-methylprop-2-enylidene]-4-oxo-2-thioxo-3-thiazolidinyl]acetic acid or a derivative thereof; 9-methyl-2,3,7-trihydroxy-6-fluorone or a derivative thereof; or 5-O-Dicaffeoyl-epiquinic acid (3,5-DCQA) or a derivative thereof. In one embodiment, the acyclic retinoid is NIK-333 [(2E,4E,6E,10E)-3,7,11,15-tetramethyl 2,4,6,10,14-hexadecapentaenoic acid] and the AKR1B10 inhibitor is tolrestat. In one embodiment, the acyclic retinoid is NIK-333 [(2E,4E,6E,10E)-3,7,11,15-tetramethyl 2,4,6,10,14-hexadecapentaenoic acid] and the AKR1B10 inhibitor is epalrestat or 2-[(5Z)-5-[(E)-3-cyclohexyl-2-methylprop-2-enylidene]-4-oxo-2-thioxo-3-thiazolidinyl]acetic acid.

In some embodiments, a level of secretion or expression of albumin, alpha fetoprotein, or a combination therein is measured before and after the contacting of the hepatoma cell.

### Definitions

For convenience, certain terms employed herein, in the specification, examples and appended claims are collected here. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

As used herein the term “human stem cell” refers to a human cell that can self-renew and differentiate to at least one cell type. The term “human stem cell” encompasses human stem cell lines, human-derived iPS cells generated using any method, human embryonic stem cells, human pluripotent cells, human multipotent stem cells, human adult progenitor cells, and human adult stem cells. A “pluripotent stem cell” is one that can give rise to all three germ layers, i.e., endoderm, mesoderm, and ectoderm. A “multipotent cell” is one that can differentiate to several different cell types within a restricted family, subset or lineage of cells. Examples of a multipotent stem cell include hematopoietic stem cells, adipose-derived stem cells, and tissue specific progenitor cells. As used herein, the term “adult stem cell” refers to a stem cell derived from a tissue of an organism after embryonic development is complete, i.e., a non-embryonic stem cell; such cells are also known in the art as “somatic stem cells.”

As used herein, the term “somatic cell” refers to any cell other than a germ cell, a cell present in or obtained from a pre-implantation embryo, or a cell resulting from proliferation of such a cell in vitro. Stated another way, a somatic cell refers to any and all cells forming the body of an organism, as opposed to germline cells. Accordingly, a “human somatic cell” refers to a somatic cell obtained from a human. In mammals, germline cells (also known as “gametes”) are the spermatozoa and ova which fuse during fertilization to produce a cell called a zygote, from which the entire mammalian embryo develops. Every other cell type in the mammalian body—apart from the sperm and ova, and the cells from which they are made (gametocytes)—is a somatic cell: internal organs, skin, bones, blood, and connective tissue are all made up of somatic cells. In some embodiments the somatic cell is a “non-embryonic somatic cell,” by which is meant a somatic cell that is not present in or obtained from an embryo and does not result from proliferation of such a cell in vitro. In some embodiments the somatic cell is an “adult somatic cell,” by which is meant a cell that is present in or obtained from an organism other than an embryo or a fetus or results from proliferation of such a cell in vitro. Unless otherwise indicated, the methods for reprogramming a somatic cell can be performed both in vivo and in vitro (where in vivo is practiced when a somatic cell is present within a subject, and where in vitro is practiced using isolated differentiated cells maintained in culture). In some embodiments, where a somatic cell or population of somatic cells are cultured in vitro, the somatic cell can be cultured in an organotypic slice culture, such as described in, e.g., meneghel-Rozzo et al., (2004), Cell Tissue Res, 316(3);295-303, which is incorporated herein in its entirety by reference.

As used herein, the term “corresponding somatic cell” or “reference somatic cell” refers to a somatic cell in culture that serves as a reference or control for the measurement of one or more properties of a somatic cell manipulated according to the compositions and methods described herein.

As used herein, the term “morphology” is used to describe one or more characteristics regarding the physical appearance of a cell that distinguishes it from or renders it similar to a given cell type or state.

As used herein, the term “reprogramming factor ” refers to a protein or other molecule or agent, such as small molecule compound or agent, that promotes or contributes to cell reprogramming to an induced pluripotent stem cell phenotype, e.g., in vitro. A reprogramming factor is added exogenously or ectopically to the cell, e.g., by direct introduction of a protein or small molecule; or by expressing the factor from a vector or heterologous construct introduced to the cell, or otherwise introducing nucleic acid (DNA or RNA) encoding the factor. The reprogramming factor is preferably, but not necessarily, from the same species as the cell being reprogrammed, i.e., human reprogramming factors for human cells. Non-limiting examples of reprogramming factors typically used for reprogramming somatic cells to pluripotency in vitro are Oct4, Nanog, Sox2, Lin28, Klf4, c-Myc, and any gene/protein or molecule that can substitute for one or more of these in a method of reprogramming somatic cells in vitro as described herein. “Reprogramming to a pluripotent state in vitro” is used herein to refer to in vitro reprogramming methods that do not require and typically do not include nuclear or cytoplasmic transfer or cell fusion, e.g., with oocytes, embryos, germ cells, or pluripotent cells.

As used herein, the term “small molecule” refers to a chemical agent including, but not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. Small molecule modulators, such as inhibitors of e.g., miRNA-145, can be identified from within a small molecule library, which can be obtained from commercial sources or from libraries as known in the art.

The term “phenotype” refers to one or a number of total biological characteristics that define a cell or organism under a particular set of environmental conditions and factors, regardless of the actual genotype.

As used herein, the term “transcription factor” refers to a protein that binds to specific parts of DNA using DNA binding domains and is part of the system that controls the transfer (or transcription) of genetic information from DNA to RNA.

The term “exogenous” as used herein refers to a nucleic acid or a protein that has been introduced by a process involving the hand of man into a biological system such as a cell or organism in which it is not normally found or in which it is found in lower amounts. A substance will be considered exogenous if it is introduced into a cell or an ancestor of the cell that inherits the substance. In contrast, the term “endogenous” refers to a substance that is native to the biological system or cell (e.g., differentiated cell).

The term “isolated cell” as used herein refers to a cell that has been removed from an organism in which it was originally found or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to a population of cells as used herein refers to a population of cells that has been removed and separated from a mixed or heterogeneous population of cells. In some embodiments, an isolated population is a substantially pure population of cells as compared to the heterogeneous population from which the cells were isolated or enriched from. In some embodiments, the isolated population is an isolated population of reprogrammed cells which is a substantially pure population of reprogrammed cells, as compared to a heterogeneous population of cells comprising reprogrammed cells and cells from which the reprogrammed cells were derived.

The term “substantially pure,” with respect to a particular cell population, refers to a population of cells that is at least about 75%, preferably at least about 85%, more preferably at least about 90%, and most preferably at least about 95% pure, with respect to the cells making up a total cell population. Recast, the terms “substantially pure” or “essentially purified,” with regard to a population of reprogrammed cells, refers to a population of cells that contain fewer than about 20%, more preferably fewer than about 15%, 10%, 8%, 7%, most preferably fewer than about 5%, 4%, 3%, 2%, 1%, or less than 1%, of cells that are not reprogrammed cells or their progeny as defined by the terms herein. In some embodiments, the methods described herein further comprise methods to expand a population of reprogrammed cells, such as ChiPS cells, wherein the expanded population of reprogrammed cells is a substantially pure population of reprogrammed ChiPS cells.

As used herein, “proliferating” and “proliferation” refer to an increase in the number of cells in a population (growth) by means of cell division. Cell proliferation is generally understood to result from the coordinated activation of multiple signal transduction pathways in response to the environment, including growth factors and other mitogens. Cell proliferation may also be promoted by release from the actions of intra- or extracellular signals and mechanisms that block or negatively affect cell proliferation.

The terms “enriching” or “enriched” are used interchangeably herein and mean that the yield (fraction) of cells of one type is increased by at least 10% over the fraction of cells of that type in the starting culture or preparation.

The term “modulate” is used consistently with its use in the art, e.g., meaning to cause or facilitate a qualitative or quantitative change, alteration, or modification in a process, pathway, or phenomenon of interest. Without limitation, such change may be an increase, decrease, or change in relative strength or activity of different components or branches of the process, pathway, or phenomenon. A “modulator” is an agent that causes or facilitates a qualitative or quantitative change, alteration, or modification in a process, pathway, or phenomenon of interest.

The terms “decrease,” “reduced,” “reduction,” “decrease,” or “inhibit” are all used herein generally to mean a decrease by a statistically significant amount. However, for avoidance of doubt, “reduced,” “reduction,” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased” ,“increase” or “enhance” or “activate” are all used herein to generally mean an increase by a statically significant amount; for the avoidance of any doubt, the terms “increased,” “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold, or at least about a 10-fold increase, or at least about a 25-fold increase, or at least about a 50-fold increase, or at least about a 100-fold increase, or any increase greater than 100-fold as compared to a reference level.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

As used herein, the term “DNA” is defined as deoxyribonucleic acid.

The term “polynucleotide” is used herein interchangeably with “nucleic acid” to indicate a polymer of nucleosides. Typically a polynucleotide of this invention is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (e.g. the succession of letters used as abbreviations for bases) that biochemically characterizes a specific nucleic acid. A polynucleotide sequence presented herein is presented in a 5′ to 3′ direction unless otherwise indicated.

The terms “polypeptide” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a nonpolypeptide moiety covalently or noncovalently associated therewith is still considered a “polypeptide”. Exemplary modifications include glycosylation and palmitoylation. Polypeptides may be purified from natural sources, produced using recombinant DNA technology, synthesized through chemical means such as conventional solid phase peptide synthesis, etc. The term “polypeptide sequence” or “amino acid sequence” as used herein can refer to the polypeptide material itself and/or to the sequence information (e.g., the succession of letters or three letter codes used as abbreviations for amino acid names) that biochemically characterizes a polypeptide. A polypeptide sequence presented herein is presented in an N-terminal to C-terminal direction unless otherwise indicated.

Any of the signaling pathways described herein can be inhibited or activated with peptide bases molecules, e.g., peptide based ligands, antibodies (e.g., monoclonal and polyclonal antibodies) and antibody fragments. Techniques for the production and isolation of antibodies and antibody fragments are well known to one of ordinary skill in the art.

“Antibodies” that can be used according to the methods described herein, such as activating TGF-β signaling pathways, include complete immunoglobulins, antigen binding fragments of immunoglobulins, as well as antigen-binding fragments that comprise antigen binding domains of immunoglobulins. “Antigen-binding fragments” of immunoglobulins include, for example, Fab, Fab′, F(ab′)2, scFv and dAbs. Modified antibody formats have been developed which retain binding specificity, but have other characteristics that may be desirable, including for example, bispecificity, multivalence (more than two binding sites), and compact size (e.g., binding domains alone).

Antibodies for use in the methods described herein can be obtained from commercial sources such as AbCam (Cambridge, Mass.), New England Biolabs (Ipswich, Mass.), Santa Cruz Biotechnologies (Santa Cruz, Calif.), Biovision (Mountain View, Calif.), R&D Systems (Minneapolis, Minn.), and Cell Signaling (Danvers, Mass.), among others. Antibodies can also be raised against a polypeptide or portion of a polypeptide by methods known to those skilled in the art. Antibodies are readily raised in animals such as rabbits or mice by immunization with the gene product, or a fragment thereof. Immunized mice are particularly useful for providing sources of B cells for the manufacture of hybridomas, which in turn are cultured to produce large quantities of monoclonal antibodies. Antibody manufacture methods are described in detail, for example, in Harlow et al., Eds., Antibodies: A Laboratory Manual, Cold Spring Harbor Laboratory, N.Y. (1988), which is hereby incorporated by reference in its entirety.

While both polyclonal and monoclonal antibodies can be used in the methods described herein, it is preferred that a monoclonal antibody is used where conditions require increased specificity for a particular protein.

The term “antibody” describes an immunoglobulin whether natural or partly or wholly synthetically produced. Antibody includes any immunoglobulin, including antibodies and fragments thereof, that binds a specific epitope. The term encompasses polyclonal, monoclonal, recombinant, humanized, and chimeric antibodies. The term also covers any polypeptide or protein having a binding domain which is, or is homologous to, an antibody binding domain. CDR grafted antibodies are also contemplated by this term.

As antibodies can be modified in a number of ways, the term “antibody” should be construed as covering any specific binding member or substance having a binding domain with the required specificity. Thus, this term covers antibody fragments, derivatives, functional equivalents and homologues of antibodies, including any polypeptide comprising an immunoglobulin binding domain, whether natural or wholly or partially synthetic. Chimeric molecules comprising an immunoglobulin binding domain, or equivalent, fused to another polypeptide are therefore included. Cloning and expression of chimeric antibodies are described in EP-A-0120694 and EP-A-0125023 and U.S. Pat. Nos. 4,816,397 and 4,816,567.

It has been shown that antigen-binding fragments of a whole antibody can perform the function of binding antigens. Examples of antigen-binding fragments for use with the compositions and methods described herein are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward, E. S. et al, Nature 341, 544-546 (1989)) which consists of a VH domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al, Science, 242, 423-426, 1988; Huston et al, PNAS USA, 85, 5879-5883, 1988); (viii) multivalent antibody fragments (scFv dimers, trimers and/or tetramers (Power and Hudson, J Immunol. Methods 242: 193-204 9 (2000)) (ix) bispecific single chain Fv dimers (PCT/US92/09965) and (x) “diabodies”, multivalent or multispecific fragments constructed by gene fusion (WO94/13804; P. Holliger et al Proc. Natl. Acad. Sci. USA 90 6444-6448, (1993)).

An “antibody combining site” is that structural portion of an antibody molecule comprised of light chain or heavy and light chain variable and hypervariable regions that specifically binds antigen.

Antibodies can also be bispecific, wherein one binding domain of the antibody is a binds to a specific target as described herein, and the other binding domain has a different specificity, e.g. to recruit an effector function, or to target the antibody to a desired loctaion, or the like.

Fab and F(ab′)₂ portions of antibody molecules can be prepared by the proteolytic reaction of papain and pepsin, respectively, on substantially intact antibody molecules by methods that are well-known. See for example, U.S. Pat. No. 4,342,566 to Theofilopolous et al. Fab′ antibody molecule portions are also well-known and are produced from F(ab′)₂ portions followed by reduction of the disulfide bonds linking the two heavy chain portions as with mercaptoethanol, and followed by alkylation of the resulting protein mercaptans with a reagent such as iodoacetamide. An antibody containing intact antibody molecules is preferred herein.

The phrase “monoclonal antibody” in its various grammatical forms refers to an antibody having only one species of antibody combining site capable of immunoreacting with a particular antigen. A monoclonal antibody thus typically displays a single binding affinity for any antigen with which it immunoreacts. A monoclonal antibody can also contain an antibody molecule having a plurality of antibody combining sites, each immunospecific for a different antigen; e.g., a bispecific (chimeric) monoclonal antibody.

The term “antigen binding domain” describes the part of an antibody which comprises the area which specifically binds to and is complementary to part or all of an antigen. Where an antigen is large, an antibody can bind to a particular part of the antigen only, which part is termed an epitope. An antigen binding domain may be provided by one or more antibody variable domains. Preferably, an antigen binding domain comprises an antibody light chain variable region (VL) and an antibody heavy chain variable region (VH).

Agonists and inhibitors for use in the present invention can alternatively be peptide or RNA aptamers. Such aptamers can for example interact with the extracellular or intracellular domains of the molecules, e.g., receptors, of interest in cells. An aptamer that interacts with the extracellular domain is preferred as it would not be necessary for such an aptamer to cross the plasma membrane of the target cell. An aptamer could also interact with a ligand such that ligands ability to interact with its receptor is inhibited. Methods for selecting an appropriate aptamer are well known in the art.

Inhibitors for use in the present invention can alternatively be based on oligonucleotides such as antisense oligonucleotides, single and double stranded siRNAs, ribozymes and decoy oligonucleotides. Oligonucleotides, such as antisense and siRNAs, act to directly block the translation of mRNA by binding thereto and thus preventing protein translation or increasing mRNA degradation, thus decreasing the levels of a receptor or other component of the signaling pathway, and thus activity, in a cell. Methods for using antisense and siRNAs for specifically inhibiting gene expression of genes whose sequence is known are well known in the art.

“RNA interference (RNAi)” is an evolutionally conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see Coburn, G. and Cullen, B., J. of Virology 76(18):9225 (2002)), thereby inhibiting expression of the target gene. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex (termed “RNA induced silencing complex,” or “RISC”) that recognizes and cleaves target mRNAs. RNAi can also be initiated by introducing nucleic acid molecules, e.g., synthetic siRNAs or RNA interfering agents, to inhibit or silence the expression of target genes, such as microRNA-145, or AKR1B10. As used herein, “inhibition of target gene expression” includes any decrease in expression or protein activity or level of the target gene, or protein encoded by the target gene, as compared to a situation wherein no RNA interference has been induced. The decrease can be of at least 20%, 30%, 40%, 50%, 60%, 70%, 80%, 90%, 95%, 99%, or more as compared to the expression of a target gene, or the activity or level of the protein encoded by a target gene which has not been targeted by an RNA interfering agent.

“Short interfering RNA” (siRNA), also referred to herein as “small interfering RNA” is defined as a nucleic acid-comprising agent which functions to inhibit expression of a target gene, by RNAi. An siRNA can be chemically synthesized, can be produced by in vitro transcription, or can be produced within a host cell. In one embodiment of the aspects described herein, an siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, 22, or 23 nucleotides in length, and can contain a 3′ and/or 5′ overhang on each strand having a length of about 0, 1, 2, 3, 4, or 5 nucleotides. The length of the overhang is independent between the two strands, e.g., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA).

siRNAs also include small hairpin (also called stem loop) RNAs (shRNAs). In one embodiment of the aspects described herein, these shRNAs are composed of or comprise a short (e.g., about 19 to about 25 nucleotide) antisense strand, followed by a nucleotide loop of about 5 to about 9 nucleotides, and the analogous sense strand. Alternatively, the sense strand can precede the nucleotide loop structure and the antisense strand can follow. These shRNAs can be encoded by plasmids, retroviruses, and lentiviruses and expressed from, for example, the pol III U6 promoter, or another promoter (see, e.g., Stewart, et al., RNA April; 9(4):493-501 (2003), incorporated by reference herein in its entirety).

The target gene or sequence of the RNA interfering agent can be a cellular gene or genomic sequence, e.g. the sequence encoding microRNA-145. An siRNA can be substantially homologous to the target gene or genomic sequence, or a fragment thereof. As used in this context, the term “homologous” is defined as being substantially identical, sufficiently complementary, or similar to the target RNA, or a fragment thereof, to effect RNA interference of the target. In addition to native RNA molecules, RNA suitable for inhibiting or interfering with the expression of a target sequence include RNA derivatives and analogs. Preferably, in some embodiments of the aspects described herein, the siRNA is identical in sequence to its target, e.g., the sequence encoding microRNA-145.

The siRNA preferably targets only one sequence. Each of the RNA interfering agents, such as siRNAs, can be screened for potential off-target effects by, for example, expression profiling. Such methods are known to one skilled in the art and are described, for example, in Jackson et al., Nature Biotechnology 6:635-637 (2003). In addition to expression profiling, one can also screen the potential target sequences for similar sequences in the sequence databases to identify potential sequences which may have off-target effects. For example, according to Jackson et al. (Id.) 15, or perhaps as few as 11 contiguous nucleotides, of sequence identity are sufficient to direct silencing of non-targeted transcripts. Therefore, one can initially screen the proposed siRNAs to avoid potential off-target silencing using sequence identity analysis by any known sequence comparison methods, such as BLAST.

siRNA molecules need not be limited to those molecules comprising only RNA, but, for example, further encompasses chemically modified nucleotides and non-nucleotides that effect RNA interference, and also include molecules wherein a ribose sugar molecule is substituted for another sugar molecule or a molecule which performs a similar function. Moreover, a non-natural linkage between nucleotide residues can be used, such as a phosphorothioate linkage. The RNA strand can be derivatized with a reactive functional group or a reporter group, such as a fluorophore. Particularly useful derivatives are modified at a terminus or termini of an RNA strand, typically the 3′ terminus of the sense strand. For example, the 2′-hydroxyl at the 3′ terminus can be readily and selectively derivatized with a variety of groups.

Other useful RNA derivatives incorporate nucleotides having modified carbohydrate moieties, such as 2′O-alkylated residues or 2′-O-methyl ribosyl derivatives and 2′-O-fluoro ribosyl derivatives. The RNA bases can also be modified. Any modified base useful for inhibiting or interfering with the expression of a target sequence can be used. For example, halogenated bases, such as 5-bromouracil and 5-iodouracil can be incorporated. The bases can also be alkylated, for example, 7-methylguanosine can be incorporated in place of a guanosine residue. Non-natural bases that yield successful inhibition can also be incorporated.

The most preferred siRNA modifications for the aspects described herein include 2′-deoxy-2′-fluorouridine or locked nucleic acid (LNA) nucleotides and RNA duplexes containing either phosphodiester or varying numbers of phosphorothioate linkages. Such modifications are known to one skilled in the art and are described, for example, in Braasch et al., Biochemistry 42: 7967-7975 (2003). Most of the useful modifications to the siRNA molecules can be introduced using chemistries established for antisense oligonucleotide technology. Preferably, the modifications involve minimal 2′-O-methyl modification, preferably excluding such modification. Modifications also preferably exclude modifications of the free 5′-hydroxyl groups of the siRNA.

siRNAs useful for targeting, for example, AKR1B10 or microRNA-145 expression can be readily designed and tested. Chalk et al. (Nucl. Acids Res. 33: D131-D134 (2005)) describe a database of siRNA sequences and a predictor of siRNA sequences. Linked to the sequences in the database is information such as siRNA thermodynamic properties and the potential for sequence-specific off-target effects. The database and associated predictive tools enable the user to evaluate an siRNA's potential for inhibition and non-specific effects. The database is available at on the world wide web at siRNA.cgb.ki.se.

Synthetic siRNA molecules, including shRNA molecules, can be obtained using a number of techniques known to those of skill in the art. For example, the siRNA molecule can be chemically synthesized or recombinantly produced using methods known in the art, such as using appropriately protected ribonucleoside phosphoramidites and a conventional DNA/RNA synthesizer (see, e.g., Elbashir, S. M. et al., Nature 411:494-498 (2001); Elbashir, S. M., et al., Genes & Development 15:188-200 (2001); Harborth, J. et al., J. Cell Science 114:4557-4565 (2001); Masters, J. R. et al., Proc. Natl. Acad. Sci., USA 98:8012-8017 (2001); and Tuschl, T. et al., Genes & Development 13:3191-3197 (1999)). Alternatively, several commercial RNA synthesis suppliers are available including, but not limited to, Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). As such, siRNA molecules are not overly difficult to synthesize and are readily provided in a quality suitable for RNAi. In addition, dsRNAs can be expressed as stem loop structures encoded by plasmid vectors, retroviruses and lentiviruses (Paddison, P. J. et al., Genes Dev. 16:948-958 (2002); McManus, M. T. et al., RNA 8:842-850 (2002); Paul, C. P. et al., Nat. Biotechnol. 20:505-508 (2002); Miyagishi, M. et al., Nat. Biotechnol. 20:497-500 (2002); Sui, G. et al., Proc. Natl. Acad. Sci., USA 99:5515-5520 (2002); Brummelkamp, T. et al., Cancer Cell 2:243 (2002); Lee, N. S., et al., Nat. Biotechnol. 20:500-505 (2002); Yu, J. Y., et al., Proc. Natl. Acad. Sci., USA 99:6047-6052 (2002); Zeng, Y., et al., Mol. Cell 9:1327-1333 (2002); Rubinson, D.A., et al., Nat. Genet. 33:401-406 (2003); Stewart, S. A., et al., RNA 9:493-501 (2003)).

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

### BRIEF DESCRIPTION OF THE FIGURES

FIG. 1 shows pluripotency-associated genes during treatment of human liver progenitor cells. Total RNA was prepared after various periods of time (6, 12, 24, 48 and 96 hr) and analyzed by reverse transcription-polymerase chain reaction for the stem cell-associated gene CD133, embryonic and pluripotency-associated genes (Oct3/4, Nanog, Klf4 and Sox2) and. Loading control, β-actin.

FIG. 2 shows a representative image of hES cell-like colony (ChiPS cells as human iPS cells).

FIG. 3 shows representative immunocytochemistry data for SSEA-4, TRA-1-60, TRA-1-81, and Nanog (human ChiPS cells as human iPS cell clone). Nuclei were stained with Hoechst 33342. Bars=100 mm.

FIGS. 4A-4B show mRNA and protein expression date of ES-cell marker genes. FIG. 4A depicts RT-PCR analysis of ES cell-marker genes in human ChiPS cells. FIG. 4B shows Western blot analysis of ES cell-marker genes in human ChiPS cells.

FIG. 5 depicts data from methylation analysis of Oct3/4 and Nanog promoter regions in human iPS cells. Bisulfite gemomic sequencing of the promoter region of Oct3/4 and Nanog in ChiPS cells, and human ES cells was performed. The open circles indicate unmethylated CpG dinucleotides, while the closed circles indicate methylated CpGs. Top Left; Oct3/4 (human ES), Top Right; Oct3/4 (human ChiPS); Bottom Left; Nanog (human ES), Bottom Right; Nanog (human ChiPS)

FIG. 6 shows teratoma formation in human ChiPS cells.

FIG. 7 demonstrates in vitro differentiation of human ChiPS cells into all three germ layers. Quantitative PCR analyses of all three germ layer markers from differentiated ChiPS cells after directed differentiation: mesoderm (HAND1, FOXF1), endoderm (AFP, GATA6, SOX17) and ectoderm (PAX6, SOX1, NCAM1). Data denote beta-actin-normalized old changes relative to undifferentiated parental iPS cells.

FIG. 8 shows chromosomal G-band analysis in typical human ChiPS cells.

FIG. 9 depicts risk evaluation of malignant transformation for different human ChiPS cell lines. 1: Human iPS cells that carry mutant p53 (iPS cells established according to the methods of the References 3, 4, 5, 6); 2: Human ChiPS cells established by only small molecules as described herein. 3: Human iPS cells that carry mutant p53 treated with PRIMA-1; 4: Human iPS cells that carry wild p53 (iPS cells established according to the methods of the Ref. 3, 4, 5, 6).

FIG. 10 shows expression of p53 and p21 in ChiPS cells by Western blotting. Right side: before knockdown of p21; Left side: after knockdown of p21.

FIGS. 11A-11B shows angiogenesis in teratomas derived from ChiPS cell lines. Microvessel density (MVD) per high-powered field (h.p.f.) of teratomas was quantified by human-specific anti-CD31 immunofluorescence. n=3-6; All values were mean±s.e.m.

FIGS. 12A-12G demonstrate that CD133 cells isolated from the Huh7 cell line (FIGS. 12A-12C) possess higher proliferative (FIGS. 12D and 12E) and clonogenic potential (FIGS. 12F and 12G) in vitro.

FIG. 13 shows phenotypic differentiation of human hepatoma cells to normal hepatocyte-like cells. First, CD133+ human hepatocellular carcinoma (HCC) cells that function as cancer stem cells and contribute to chemoresistance from Huh 7 cells were sorted. The CD133⁺ human HCC cells were treated with 10 μM acyclic retinoid plus 10 μM tolrestat. As a result, phenotypic differentiation of human hepatoma cells to normal hepatocyte-like cells was induced in 2 days. The normal hepatocyte-like cells from CD133⁺ human HCC cells were designated induced hepatocyte-like cells (iHep).

FIG. 14 shows phenotypic differentiation of human hepatoma cells to normal hepatocyte-like cells. Left image is human hepatoma-cells and right image is normal hepatocyte-like cells (induced hepatocyte-like cells; iHep).

FIG. 15 shows RT-PCR analysis of CD133, Oct3/4 and β-catein using CD133+ human HCC cells purified from Huh7 with or without acyclic retinoid (AR) plus AKR1B10 inhibitor (tolrestat).

FIG. 16 demonstrates generation of human induced pluripotent stem (iPS) cells from hepatocyte-like cells that were generated from human hepatoma cells. Human induced pluripotent stem (iPS) cells were generated from the hepatocyte-like cells that had been generated from human hepatoma cells, using 5′-aza-2′-deoxycytidine (5-AZAC) plus trichostatin A (TSA) without Oct3/4 and c-Myc. The iHep were maintained using a medium for Primate ES cell culture supplemented with 40 ng/ml of hepatocytes growth factor and 20 ng/ml of epidermal growth factor. Furthermore, 500 nM 5′-aza-2′ deoxycytidine (5-AZAC), 150 nM trichostatin A (TSA), 1 μM EMD 616452 as an inhibitor for Tgf beta receptor I kinase/activin-like kinase 5 (Alk5) and 0.5 mM valproic acid (VPA) were administered in the Primate ES cell medium for 96 hr. After 14 days, human iPS cells (iPS-hHepC) were established. Oct3/4 was reactivated by 5-AZAC+TSA. Furthermore, Sox2 and Klf4 were reactivated by EMD 616452 and VPA, respectively.

FIG. 17 demonstrates morphology of human induced pluripotent stem (iPS) cells generated from hepatocyte-like cells originally generated from human hepatoma cells.

FIG. 18 shows RT-PCR analysis of embryonic stem (ES) cell-marker genes in iPS-cells derived from hepatocyte-like cells (iPS-hHepC) that were generated from human hepatoma cells. Total RNA was prepared after various periods of time (6, 12, 24, 48 and 96 hr) and analyzed by RT-PCR for the stem cell-associated gene CD133, embryonic and pluripotency-associated genes (Oct3/4, Nanog, Klf4 and Sox2) and loading control, β-actin.

FIG. 19 shows Western blot analysis of ES cell-marker genes in iPS-cells derived from hepatocyte-like cells that were generated from human hepatoma cells (iPS-hHepC).

FIG. 20 shows methylation analysis of Oct3/4 and Nanog promoter regions in iPS-cells derived from hepatocyte-like cells (iPS-hHepC) that were generated from human hepatoma cells using bisulfite gemomic sequencing of the promoter regions of Oct3/4 and Nanog. The open circles indicate unmethylated CpG dinucleotides, while the closed circles indicate methylated CpGs.

FIG. 21 shows teratoma formation (muscle, neural tissue, and gut-like epithelium) in iPS-cells derived from hepatocyte-like cells (iPS-hHepC) generated from CD133+ human HCC cells.

FIG. 22 shows representative G-banded karyotype of a Huh 7 cell line. Arrowheads indicate breakpoints. M1:der(1)t(1;21)(p22;p13); M2:der(6;17)(q10;q10); M3:der(16)t(6;16)(q21;p13); M4:der(21)t(1;21)(p22;p13).

FIG. 23 shows chromosomal G-band analysis of representative iPS-cells derived from hepatocyte-like cells (iPS-hHepC) using iPS-hHepC clone A.

FIG. 24A shows expression of p53 and p21 in iPS-cells derived from hepatocyte-like cells that were generated from human hepatoma cells (iPS-hHepC) by Western blotting (Right side: before knockdown of p21, Left side: after knockdown of p21). FIG. 24B shows a comparison of expression of alfa-fetoprotein (AFP) and albumin between a knockdown of p21 group [p21 siRNA (+)] and control group [p21 siRNA (−)] by RT-PCR during the process of differentiation induction based on the protocols for the induction of differentiation from hES cells to human hepatocyte-like cells. FIG. 24C shows expression of AFP and albumin in the control group 4321 siRNA (−)] by RT-PCR after 21 days.

### DETAILED DESCRIPTION

The methods and compositions described herein are based, in part, on novel discoveries by the inventors that one or more small molecule agents can be used to reprogram adult somatic cells, and that one or more small molecule agents can be used to redifferentiate cancerous cells, such as hepatoma cells. Further, described herein are novel methods for assessing the potential of a reprogrammed cell, generated using, for example, the compositions and methods provided herein, or any other method known in the art, to undergo cancerous transformation, and methods of inhibiting the same. Thus, the methods and compositions described herein relate to methods and compositions for cellular reprogramming of somatic cells and redifferentiation of cancer cells.

One goal of regenerative or personalized medicine is to be able to convert an adult differentiated cell into other cell types for tissue repair and regeneration. Retroviral transduction with three genes: Sox2, Oct4, and Klf4, has been shown to directly reprogram mouse or human differentiated cells (e.g., somatic cells) to a pluripotent stem cell state. Unfortunately, the resulting induced pluripotent stem (iPS) cells are suboptimal for clinical uses in transplantation medicine and disease modeling because the viral transgenes they contain may spontaneously re-activate, a process that has lead to tumor formation in mice generated from iPS cells. Furthermore, in two gene therapy trials, retroviral vectors used for delivery of reprogramming genes were themselves shown to be intrinsically oncogenic (B. Feng et al., Cell Stem Cell 2009, 4: 301-312).

While generation of iPS cells using non-integrating DNA-based methods have been reported and are an improvement over retroviral delivery of reprogramming factors, use of such methods in therapeutic transplantation medicine and disease models is limited because these vectors are still considered to cause permanent alterations in chromosomal DNA that can be difficult to detect. Therefore, clinical implementation of reprogramming technology would optimally avoid viral transduction and the introduction of any transgenic DNA in general. While transduction with recombinant protein factors has been reported to be capable of reprogramming mouse embryonic fibroblasts, use of protein factors is limited due to this process is highly inefficient and too laborious and expensive to implement at a large-scale. Furthermore, this methodology requires the use of valproic acid (VPA), a histone deacetylase (HDAC) inhibitor that can cause long-lasting, heritable changes in the expression of imprinted and cancer-related genes in mammalian cells.

The identification of small molecules that can efficiently reprogram patient cells without the use of DNA expression vectors or large-scale protein preparations can reproducibly allow the efficient generation of pluripotent stem cells that would be genetically unmodified, and as a result, most suitable for use in cell therapies for regenerative medicine applications. Small molecules that globally alter chromatin structure, including the DNA methyltransferase inhibitor 5-aza-cytidine (AZA) and the HDAC inhibitor VPA, can increase reprogramming efficiency and even reduce the number of factors required for reprogramming. Treatment with these inhibitors relaxes the structure of chromatin and in turn lowers the barrier to activation of endogenous pluripotency associated genes. However, Oct4 and Sox2 not only collaborate in reprogramming by activating genes required for pluripotency, they also function to repress genes promoting differentiation. It has been shown that small molecule inhibitors of DNA methyltransferases such as 5-aza-Cytidine (5azaC) or histone deacetylases (HDACs) such as valproic acid (VPA), can increase reprogramming efficiency with all four factors or just three factors. However, in reprogramming experiments, these small molecules do not appear to replace the reprogramming factors, but instead increase their overall efficiency. Thus, there is a significant need to identify additional small molecules that can function in reprogramming either independently, or in concert with chemicals modulating chromatin structure.

### Stem/Progenitor Cells

Stem cells or progenitor cells are undifferentiated cells defined by their ability at the single cell level to both self-renew and differentiate to produce progeny cells, including self-renewing progenitors, non-renewing progenitors, and terminally differentiated cells. Stem cells or progenitor cells, depending on their level of differentiation, are also characterized by their ability to differentiate in vitro into functional cells of various cell lineages from multiple germ layers (endoderm, mesoderm and ectoderm), as well as to give rise to tissues of multiple germ layers following transplantation and to contribute substantially to most, if not all, tissues following injection into blastocysts.

Stem cells are classified by their developmental potential as: (1) totipotent, which is able to give rise to all embryonic and extraembryonic cell types; (2) pluripotent, which is able to give rise to all embryonic cell types, i.e., endoderm, mesoderm, and ectoderm; (3) multipotent, which is able to give rise to a subset of cell lineages, but all within a particular tissue, organ, or physiological system (for example, hematopoietic stem cells (HSC) can produce progeny that include HSC (self-renewal), blood cell restricted oligopotent progenitors and the cell types and elements (e.g., platelets) that are normal components of the blood); (4) oligopotent, which is able to give rise to a more restricted subset of cell lineages than multipotent stem cells; and (5) unipotent, which is able to give rise to a single cell lineage (e.g., spermatogenic stem cells).

Adult stem cells or progenitor cells are generally limited to differentiating into different cell types of their tissue of origin. However, if the starting stem cells are derived from the inner cell mass of the embryo, they can give rise to all cell types of the body derived from the three embryonic germ layers: endoderm, mesoderm and ectoderm. Stem cells with this property are said to be pluripotent. Embryonic stem cells are one kind of pluripotent stem cell. The term “embryonic stem cell” refers to those pluripotent stem cells of the inner cell mass of the embryonic blastocyst (described by Thomson et al. (1998) Science 282:1145; U.S. Pat. No. 7,615,374; U.S. Pat. No. 7,611,852; U.S. Pat. No. 7,582,479; U.S. Pat. No. 7,514,260; U.S. Pat. No. 7,439,064, U.S. Pat. No. 7,390,657; U.S. Pat. No. 7,220,584; U.S. Pat. No. 7,217,569; U.S. Pat. No. 7,148,062; U.S. Pat. No. 7,029,913; U.S. Pat. No. 6,887,706; U.S. Pat. No. 6,613,568; U.S. Pat. No. 6,602,711; U.S. Pat. No. 6,280,718; U.S. Pat. No. 6,200,806; and U.S. Pat. No. 5,843,780, which are incorporated herein by reference). Such cells can similarly be obtained from the inner cell mass of blastocysts derived from somatic cell nuclear transfer (see, for example, U.S. Pat. Nos. 5,945,577, 5,994,619, 6,235,970, which are incorporated herein by reference). The distinguishing characteristics of an embryonic stem cell define an embryonic stem cell phenotype. Accordingly, a cell has the phenotype of an embryonic stem cell if it possesses one or more of the unique characteristics of an embryonic stem cell, such that the cell can be distinguished from other cells. Exemplary distinguishing embryonic stem cell characteristics include, without limitation, gene expression profile, proliferative capacity, differentiation capacity, karyotype, responsiveness to particular culture conditions, and the like. Also included in the term ‘embryonic stem cells’ are human embryonic germ (hEG) cells (Shambloft et al., Proc. Natl. Acad. Sci. USA 95:13726, 1998).

Cells derived from embryonic sources can include embryonic stem cells or stem cell lines obtained from a stem cell bank or other recognized depository institution. Other means of producing stem cell lines include the method of Chung et al (2006) which comprises taking a blastomere cell from an early stage embryo prior to formation of the blastocyst (at around the 8-cell stage). The technique corresponds to the pre-implantation genetic diagnosis technique routinely practiced in assisted reproduction clinics. The single blastomere cell is then co-cultured with established ES-cell lines and then separated from them to form fully competent ES cell lines.

Embryonic stem cells are considered to be undifferentiated when they have not committed to a specific differentiation lineage. Such cells display morphological characteristics that distinguish them from differentiated cells of embryo or adult origin. Undifferentiated embryonic stem (ES) cells are easily recognized by those skilled in the art, and typically appear in the two dimensions of a microscopic view in colonies of cells with high nuclear/cytoplasmic ratios and prominent nucleoli.

Somatic or adult stem or progenitor cells have major practical advantages, for example, using somatic stem cells or somatic progenitor cells allows a patient's own cells to be expanded in culture and then re-introduced into the patient. The terms “adult stem cell,” “adult progenitor cell,” “somatic stem cell” or “somatic progenitor cell,” as used herein, refer to any multipotent stem cell derived from non-embryonic tissue, including fetal, juvenile, and adult tissue. Adult progenitor or stem cells have been isolated from a wide variety of adult tissues including blood, bone marrow, brain, olfactory epithelium, skin, pancreas, skeletal muscle, and cardiac muscle. Each of these adult progenitor or stem cells can be characterized based on gene expression, factor responsiveness, and morphology in culture. Exemplary adult stem cells or adult progenitor cells include neural stem cells, neural crest stem cells, mesenchymal stem cells, hematopoietic stem cells, and pancreatic stem cells. As indicated above, adult progenitor or stem cells have been found resident in virtually every tissue. In addition, induced pluripotent stem cells (iPS cells), such as reprogrammed ChiPS cells, as further discussed herein below, derived or generated from a patient sample provide a source of progenitor or stem cells that can be expanded and re-introduced to the patient, before or after stimulation to differentiate to a desired lineage of phenotype.

In some embodiments of the aspects described herein, a progenitor or stem cell is isolated. Most conventional methods to isolate a particular stem cell of interest involve positive and negative selection using markers of interest. Agents can be used to recognize stem cell markers, for instance labeled antibodies that recognize and bind to cell-surface markers or antigens on desired stem cells. Antibodies or similar agents specific for a given marker, or set of markers, can be used to separate and isolate the desired stem cells using fluorescent activated cell sorting (FACS), panning methods, magnetic particle selection, particle sorter selection and other methods known to persons skilled in the art, including density separation (Xu et al. (2002) Circ. Res. 91:501; U.S. patent application Ser. No. 20030022367) and separation based on other physical properties (Doevendans et al. (2000) J. Mol. Cell. Cardiol. 32:839-851). Alternatively, genetic selection methods can be used, where a progenitor or stem cell can be genetically engineered to express a reporter protein operatively linked to a tissue-specific promoter and/or a specific gene promoter; therefore the expression of the reporter can be used for positive selection methods to isolate and enrich the desired stem cell. For example, a fluorescent reporter protein can be expressed in the desired stem cell by genetic engineering methods to operatively link the marker protein to a promoter active in a desired stem cell (Klug et al. (1996) J. Clin. Invest. 98:216-224; U.S. Pat. No. 6,737,054). Other means of positive selection include drug selection, for instance as described by Klug et al., supra, involving enrichment of desired cells by density gradient centrifugation. Negative selection can be performed, selecting and removing cells with undesired markers or characteristics, for example fibroblast markers, epithelial cell markers etc.

Undifferentiated ES cells express genes that can be used as markers to detect the presence of undifferentiated cells. The polypeptide products of such genes can be used as markers for negative selection. For example, see U.S. application Ser. No. 2003/0224411 A1; Bhattacharya (2004) Blood 103(8):2956-64; and Thomson (1998), supra., each herein incorporated by reference. Human ES cell lines express cell surface markers that characterize undifferentiated nonhuman primate ES and human EC cells, including, but not limited to, stage-specific embryonic antigen (SSEA)-3, SSEA-4, TRA-I-60, TRA-1-81, and alkaline phosphatase. The globo-series glycolipid GL7, which carries the SSEA-4 epitope, is formed by the addition of sialic acid to the globo-series glycolipid Gb5, which carries the SSEA-3 epitope. Thus, GL7 reacts with antibodies to both SSEA-3 and SSEA-4. Undifferentiated human ES cell lines do not stain for SSEA-1, but differentiated cells stain strongly for SSEA-1. Methods for proliferating hES cells in the undifferentiated form are described in WO 99/20741, WO 01/51616, and WO 03/020920, the contents of which are herein incorporated by reference in their entireties.

In some embodiments of the aspects described herein, the methods can further comprise enrichment and isolation of stem cells. The stem cells are selected for a characteristic of interest. In some embodiments, a wide range of markers can be used for selection. One of skill in the art will be able to select markers appropriate for the desired cell type. The characteristics of interest include expression of particular markers of interest, for example, specific subpopulations of stem cells and progenitor cells express specific markers.

In some embodiments of the aspects described herein, the methods can further comprise expansion of a population of stem or progenitor cells. The cells are optionally collected, separated, and further expanded, generating larger populations of stem cells for use in making cells of a particular cell type.

### Reprogramming and ChiPS Cells

Although differentiation is generally irreversible under physiological contexts, several methods have been recently developed to reprogram somatic cells. Reprogramming methodologies, such as somatic cell nuclear transfer, have proved successful for several species, but there are technical and ethical issues in applying this approach to human cells (Yamanaka, 2007).

Accordingly, as used herein, the term “reprogramming” refers to a process that alters or reverses the differentiation state of a differentiated cell (e.g., a somatic cell). Stated another way, reprogramming refers to a process of driving the differentiation of a cell backwards to a more undifferentiated or more primitive type of cell. The cell to be reprogrammed can be either partially or terminally differentiated prior to reprogramming. In some embodiments, reprogramming encompasses complete reversion of the differentiation state of a differentiated cell (e.g., a somatic cell) to a pluripotent state. In some embodiments, reprogramming also encompasses partial reversion of the differentiation state of a differentiated cell (e.g., a somatic cell) to a multipotent state. In some embodiments, reprogramming encompasses complete or partial reversion of the differentiation state of a differentiated cell (e.g., a somatic cell) to an undifferentiated cell. Reprogramming also encompasses partial reversion of the differentiation state of a somatic cell to a state that renders the cell more susceptible to complete reprogramming to a pluripotent state when subjected to additional manipulations, such as those described herein. Such contacting can result in expression of particular genes by the cells, the expression of which further contributes to reprogramming. In certain embodiments described herein, reprogramming of a differentiated cell (e.g., a somatic cell) causes the differentiated cell to assume an undifferentiated state (e.g., is an undifferentated cell). In some embodiments, reprogramming of a differentiated cell (e.g., a somatic cell) causes the differentiated cell to assume a pluripotent-like state. The resulting cells are referred to as “reprogrammed cells,” and in those embodiments where only small molecules were used to generate the cells, then they can be referred to as “chemically induced reprogrammed stem cells” or ChIPS cells.

Reprogramming can involve alteration, e.g., reversal, of at least some of the heritable patterns of nucleic acid modification (e.g., methylation), chromatin condensation, epigenetic changes, genomic imprinting, etc., that occur during cellular differentiation. Reprogramming is distinct from simply maintaining the existing undifferentiated state of a cell that is already pluripotent or maintaining the existing less than fully differentiated state of a cell that is already a multipotent cell (e.g., a hematopoietic stem cell). Reprogramming is also distinct from promoting the self-renewal or proliferation of cells that are already pluripotent or multipotent, although the compositions and methods described herein can also be of use for such purposes, in some embodiments.

Reprogramming methodologies for generating pluripotent cells using defined combinations of transcription factors have recently been described for producing cells termed as “induced pluripotent stem (iPS) cells.” Yamanaka and Takahashi converted mouse somatic cells to ES cell-like cells with expanded developmental potential using only four transcription factors (Takahashi and Yamanaka, 2006). This induced pluripotency method was achieved by the direct transduction of Oct4, Sox2, Klf4, and c-Myc (OSKM), and serves as a robust reprogramming technique (Maherali and Hochedlinger, 2008). These iPS cells closely resemble ES cells as they restore the pluripotency-associated transcriptional circuitry and epigenetic landscape. In addition, mouse iPS cells satisfy all the standard assays for pluripotency: specifically, in vitro differentiation into cell types of the three germ layers, teratoma formation, contribution to chimeras, germline transmission (Maherali and Hochedlinger, 2008), and tetraploid complementation (Woltjen et al., 2009).

Subsequent studies have shown that human iPS cells can be obtained using similar transduction methods (Lowry et al., 2008; Park et al., 2008; Takahashi et al., 2007; Yu et al., 2007b), and the transcription factor trio, OCT4, SOX2, and NANOG, has been established as the core set of transcription factors that govern pluripotency (Jaenisch and Young, 2008). Besides extending autoregulatory and feed-forward loops, OCT4, SOX2, and NANOG also extensively cotarget downstream genes (Boyer et al., 2005; Chen et al., 2008; Kim et al., 2008a; Loh et al., 2006). The production of iPS cells is typically achieved by the introduction of nucleic acid sequences encoding stem cell-associated genes into an adult, somatic cell. Historically, these nucleic acids have been introduced using viral vectors and the expression of the gene products results in cells that are morphologically, biochemically, and functionally similar to pluripotent stem cells (e.g., embryonic stem cells).

iPS cells can be generated or derived from terminally differentiated somatic cells, as well as from adult stem cells, or somatic stem cells. That is, a non-pluripotent progenitor cell can be rendered pluripotent or multipotent by reprogramming. In such instances, it may not be necessary to include as many reprogramming factors as required to reprogram a terminally differentiated cell. Further, reprogramming can be induced by the non-viral introduction of reprogramming factors, e.g., by introducing the proteins themselves, or by introducing nucleic acids that encode the reprogramming factors, or by introducing messenger RNAs that upon translation produce the reprogramming factors (as described in Warren et al., Cell Stem Cell, 2010 Nov. 5; 7(5):618-30, the contents of which are incorporated by reference in their entireties). Reprogramming can be achieved by introducing a combination of nucleic acids encoding stem cell-associated genes including, for example Oct-4 (also known as Oct-3/4 or Pouf51), Sox1, Sox2, Sox3, Sox 15, Sox 18, NANOG, Klf1, Klf2, Klf4, Klf5, NR5A2, c-Myc, 1-Myc, n-Myc, Rem2, Tert, and LIN28. In one embodiment, reprogramming using the methods and compositions described herein can further comprise introducing one or more of Oct-3/4, a member of the Sox family, a member of the Klf family, and a member of the Myc family to a somatic cell. In one embodiment, the methods and compositions described herein further comprise one or more of each of Oct 4, Sox2, Nanog, c-MYC and Klf4 for reprogramming.

While the progress from mouse to human iPS cells has opened the possibility of autologous regenerative medicine whereby patient-specific pluripotent cells are derived from adult somatic cells, several limitations of most existing iPS cells prohibit their usage in the clinical setting (Maherali and Hochedlinger, 2008). First, virus-mediated delivery of reprogramming factors introduces unacceptable risks of permanent transgene integration into the genome. The resulting genomic alteration and possible reactivation of viral transgenes pose serious clinical concerns. Second, some reprogramming factors, such as Klf4 and c-Myc, are oncogenic. Third, iPSC reprogramming is an inefficient and slow process. During the course of reprogramming, a substantial reduction in efficiency can also result from incomplete reprogramming (Mikkelsen et al., 2008; Okita et al., 2007; Silva et al., 2008).

The efficiency of reprogramming (i.e., the number of reprogrammed cells) derived from a population of starting cells can be enhanced by the addition of various small molecules as shown by Shi, Y., et al (2008) Cell-Stem Cell 2:525-528, Huangfu, D., et al (2008) Nature Biotechnology 26(7):795-797, and Marson, A., et al (2008) Cell-Stem Cell 3:132-135, which are incorporated herein by reference in their entireties. It is contemplated that the methods and compositions described herein can also be used in combination with a single small molecule (or a combination of small molecules) that enhances the efficiency or rate of induced pluripotent stem cell production. Some non-limiting examples of agents that enhance reprogramming efficiency include soluble Wnt, Wnt conditioned media, BIX-01294 (a G9a histone methyltransferase), PD0325901 (a MEK inhibitor), DNA methyltransferase inhibitors, histone deacetylase (HDAC) inhibitors, valproic acid, 5′-azacytidine, dexamethasone, suberoylanilide, hydroxamic acid (SAHA), and trichostatin (TSA), among others.

To confirm the induction of pluripotent stem cells using the compositions and methods described herein, isolated clones can be tested for the expression of a stem cell marker. Such expression identifies the cells as induced pluripotent stem cells. Stem cell markers can be selected from the non-limiting group including SSEA3, SSEA4, CD9, Nanog, Fbx15, Ecat1, Esg1, Eras, Gdf3, Fgf4, Cripto, Dax1, Zpf296, Slc2a3, Rex1, Utf1, and Nat1. Methods for detecting the expression of such markers can include, for example, RT-PCR and immunological methods that detect the presence of the encoded polypeptides, such as Western blots or flow cytometric analyses. In preferred embodiments, detection does not involve only RT-PCR, but also includes detection of protein markers.

The pluripotent stem cell character of isolated cells can be confirmed by any of a number of tests evaluating the expression of ES markers and the ability to differentiate to cells of each of the three germ layers. As one example, teratoma formation in nude mice can be used to evaluate the pluripotent character of the isolated clones. The cells are introduced to nude mice and histology and/or immunohistochemistry is performed on a tumor arising from the cells. The growth of a tumor comprising cells from all three germ layers, for example, further indicates that the cells are pluripotent stem cells.

### Compositions, Methods and Kits for Generation of ChiPS Cells

Described herein are cell-growth compositions comprising one or more modulating agents specific for microRNA-145 and the TGF-β signaling pathway, and methods using such cell-growth compositions, for the generation of reprogrammed cells, termed herein as “chemically induced pluripotent stem cells” (i.e., “ChiPS cells). Generally, in preferred embodiments, these compositions and methods do not comprise the use of exogenous transcription factors or nucleic acid sequences encoding exogenous transcription factors, such as OCT3/4 or SOX-2.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments of the aspects described herein, agents include nucleic acids, nucleic acid analogues, proteins, antibodies, peptides, aptamers, oligomers of nucleic acids, amino acids, or carbohydrates, and includes without limitation proteins, oligonucleotides, ribozymes, DNAzymes, glycoproteins, siRNAs, lipoproteins, aptamers, and modifications and combinations thereof, etc. In certain embodiments, agents are small molecules having a chemical moiety. For example, chemical moieties include unsubstituted or substituted alkyl, aromatic, or heterocyclyl moieties including macrolides, leptomycins and related natural products or analogues thereof. Compounds can be known to have a desired activity and/or property, or can be selected from a library of diverse compounds. Accordingly, as used herein a “modulating agent” refers to any compound or substance that causes or facilitates a qualitative or quantitative change, alteration, or modification in a process, pathway, or phenomenon of interest. For example, in some embodiments of the aspects described herein, a modulating agent can increase or promote a process, pathway, or phenomenon of interest, and can act as an agonist. In other embodiments of the aspects described herein, a modulating agent can decrease, inhibit, or reduce a process, pathway, or phenomenon of interest, and can act as an antagonist.

Accordingly, in one aspect, provided herein are cell-growth compositions comprising one or more modulating agents specific for microRNA-145 and one or more modulating agents specific for one or more components of the TGF-β signaling pathway. In some embodiments of the aspects described herein, the cell-growth compositions comprise an inhibitory agent specific for microRNA-145, such as an antagonist. In some embodiments of the aspects described herein, the cell-growth compositions comprise an activating agent specific for the TGF-β signaling pathway, such as a TGF-β signaling pathway agonist.

As used herein, the term “cell-growth composition” refers to a composition comprising one or more agents, such as agonists or inhibitors of one or more biological pathways, for modulating the growth of a cell to which the cell-growth composition is added or contacted with. In some embodiments of the aspects described herein, a cell that is contacted with a cell-growth composition is an adult somatic cell, and contacting of the cell with the cell-growth composition causes reprogramming of the cell to a pluripotent state. In some embodiments of the aspects described herein, a cell-growth composition can be added to or formulated with a “cell culture medium.” A “cell culture medium” (also referred to herein as a “culture medium” or “medium”) as referred to herein refers to a medium for culturing cells comprising nutrients that maintain cell viability and support proliferation. The cell culture medium can contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types, such as human stem or progenitor cells, are known to those skilled in the art.

In another aspect, provided herein are compositions comprising a human somatic cell in admixture with one or more modulating agents specific for microRNA-145 and one or more modulating agents specific for the TGF-β signaling pathway. In some embodiments of the aspects described herein, the compositions comprise an inhibitory agent specific for microRNA-145, such as an antagonist. In some embodiments of the aspects described herein, the compositions comprise an activating agent specific for TGF-β signaling, such as a TGF-β signaling pathway agonist.

In some embodiments of these aspects, a human somatic cell for use with the compositions and methods described herein has endogenous expression of OCT3/4. In other embodiments of these aspects, a human somatic cell for use with the compositions and methods described herein has endogenous expression of SOX2. In some embodiments of these aspects, the human somatic cell has endogenous expression of both OCT3/4 and SOX2.

As used herein, “expression” of a molecule, such as an RNA or protein, refers to the cellular processes involved in producing RNA and proteins and as appropriate, secreting proteins, and includes, for example, transcription, translation, folding, modification and processing of a given molecule encoded by a given gene. “Expression products” include RNA transcribed from a gene, and polypeptides obtained by translation of mRNA transcribed from a gene.

Accordingly, as used herein, the terms “endogenous expression” or “endogenously expressed” refers to expression of a gene (transcriptional expression into RNA and/or translational expression into protein) in a cell under normal steady state and/or developmental conditions, in the absence of any exogenous factors or manipulation, such that in its native, unmanipulated state, the cell expresses the RNA product or protein product in question. For example, OCT3/4 is a gene that is “endogenously expressed” in human embryonic stem cells. Endogenous expression of a gene can depend, for example, on the developmental stage of a cell, such that a cell at a particular differentiation state endogenously expresses a gene, but at later or earlier differentiation states does not have endogenous expression of the gene. Accordingly, a gene that is endogenously expressed in a somatic cell has expression of that gene at a level at least 5% higher, at least 10% higher, at least 15% higher, at least 20% higher, at least 30% higher, at least 40% higher, at least 50% higher, at least 60% higher, at least 70% higher, at least 80% higher, at least 90% higher, at least 100% higher, at least 2-fold higher, at least 5-fold higher, at least 10-fold higher, at least 25-fold higher, at least 50-fold higher, at least 100-fold higher, at least 1000-fold higher, or more than expression of the gene in a somatic cell that does not have endogenous expression of a gene. Accordingly, as used herein, when a somatic cell “does not have endogenous expression” of a gene, it indicates that the RNA and/or protein product of the gene cannot be detected in that cell using standard techniques known to one of skill in the art.

Assessment and/or determination of whether a cell has endogenous expression of a given molecule or gene can be determined using methods known to one of skill in the art, such as quantitative RT-PCT (qRT-PCR) for detection of endogenous RNA expression, and/or Western blot analyses for detection of endogenous protein expression. In preferred embodiments, the human somatic cell having endogenous expression of OCT3/4 or SOX-2 refers to endogenous protein expression of these molecules.

In other aspects, provided herein are methods of generating chemically induced pluripotent stem (ChiPS) cells. In such methods, a human somatic cell is contacted with one or more inhibitors of microRNA-145, and one or more activators or agonists of TGF-β signaling, thus generating a chemically induced pluripotent stem cell.

In some embodiments of these aspects, the human somatic cell has endogenous expression of OCT3/4. In other embodiments of these aspects, the human somatic cell has endogenous expression of SOX2. In some embodiments of these aspects, the human somatic cell has endogenous expression of both OCT3/4 and SOX2.

The terms “contacting” or “contact” as used in connection with the compositions and methods described herein refer to contacting a human somatic cell with any composition described herein and includes subjecting the cell to a culture media which comprises that composition, such as a culture media comprising the cell-growth compositions comprising one or more inhibitors of microRNA-145, and one or more activators of TGF-β signaling. In those embodiments where the human somatic cell to be contacted is in vivo, contacting the human somatic cell with the composition includes administering the composition, and any other pharmaceutically acceptable carrier(s), to a subject via an appropriate administration route such that the composition contacts the human somatic cell in vivo.

### Somatic Cell Types for Reprogramming

The methods described herein can be used, e.g., to chemically reprogram a human somatic cell to a pluripotent state. Such somatic cells can be obtained, for example from a patient, to prepare patient-specific stem cells (e.g., patient-specific pluripotent stem or progenitor cells). A variety of somatic cells can be used, such as, hair follicle cells, a cell from a blood sample, a cell from adipose tissue, a stomach cell, a liver cell, or a cell from skin (e.g., fibroblast or other cell type, e.g., keratinocyte, melanocyte, Langerhans cell, or Merkel cell).

Somatic cells, as used herein, refer to any cells forming the body of an organism, as opposed to germline cells. In mammals, germline cells (also known as gametes) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a differentiated somatic cell. For example, internal organs, skin, bones, blood, and connective tissue are all made up of differentiated somatic cells.

Additional somatic cell types for use with the compositions and methods described herein include: a fibroblast (e.g., a primary fibroblast), a muscle cell (e.g., a myocyte), a cumulus cell, a neural cell, a mammary cell, a hepatocyte and a pancreatic islet cell. In some embodiments, the somatic cell is a primary cell line or is the progeny of a primary or secondary cell line. In some embodiments, the somatic cell is obtained from a human sample, e.g., a hair follicle, a blood sample, a biopsy (e.g., a skin biopsy or an adipose biopsy), a swab sample (e.g., an oral swab sample), and is thus a human somatic cell.

Some non-limiting examples of differentiated somatic cells include, but are not limited to, epithelial, endothelial, neuronal, adipose, cardiac, skeletal muscle, immune cells, hepatic, splenic, lung, circulating blood cells, gastrointestinal, renal, bone marrow, and pancreatic cells. In some embodiments, a somatic cell can be a primary cell isolated from any somatic tissue including, but not limited to brain, liver, lung, gut, stomach, intestine, fat, muscle, uterus, skin, spleen, endocrine organ, bone, etc. Further, the somatic cell can be from any mammalian species, with non-limiting examples including a murine, bovine, simian, porcine, equine, ovine, or human cell. In preferred embodiments, the somatic cell is a human somatic cell.

When the reprogrammed cells (e.g, chemical human induced pluripotent stem cells) are used for therapeutic treatment of diseases, it is desirable to use somatic cells isolated from the patient being treated. For example, somatic cells involved in diseases, somatic cells participating in therapeutic treatment of diseases and the like can be used. In some embodiments, a method for selecting the reprogrammed cells from a heterogeneous population comprising reprogrammed cells and somatic cells they were derived or generated from can be performed by any well-known means, for example, a drug resistance gene or the like, such as selectable marker genes can be used as a marker gene to isolate the reprogrammed cells using the selectable marker as index.

Where the cell is maintained under in vitro conditions, conventional tissue culture conditions and methods can be used, and are known to those of skill in the art. Isolation and culture methods for various cells are well within the abilities of one skilled in the art. Various media that can maintain undifferentiated state and pluripotency of ES cells and various media which cannot maintain such properties are known in this field, and reprogrammed cells generated using the compositions and methods as described here can be efficiently isolated by using a combination of appropriate media. Differentiation and proliferation abilities of the reprogrammed cells can be easily confirmed by those skilled in the art by using confirmation means widely applied to ES cells.

In some embodiments of the aspects, chemically induced pluripotent stem cells (e.g. ChiPS cell) generated using the methods and compositions described herein enable the generation of patient- or disease-specific reprogrammed cells, without the need for genetically manipulating the cells (e.g. in the absence of using viral means or other genetic manipulation methods to increase the expression of reprogramming transcription factors). The ChiPS cells as described herein are indistinguishable from ES cells in morphology, proliferation, gene expression, and teratoma formation. Furthermore, as described herein in the Examples, when transplanted into blastocysts, such chemically-induced mouse iPS cells can give rise to adult chimeras, which are competent for germline transmission (Maherali et al., Cell Stem Cell 1:55-70, 2007; Okita et al., Nature 448:313-17, 2007; Wemig et al., Nature 448:318-324, 2007). ChiPS cells are also expandable and indistinguishable from human embryonic stem (ES) cells in morphology and proliferation. Furthermore, these chemically-induced reprogrammed cells can differentiate into cell types of the three germ layers in vitro and in teratomas,

### MicroRNA-145

In some embodiments of the aspects described herein, the compositions and methods for generating a ChiPS cell comprise at least one inhibitory agent specific for microRNA-145 or miRNA145, such as an nucleic acid sequence that specifically binds to and inhibits microRNA-145, e.g., a nucleic acid inhibitor of microRNA-145, or a small molecule inhibitor of microRNA-145. In some such embodiments, the nucleic acid inhibitor of microRNA-145 is a anti-microRNA-145 LNA oligonucleotide or a 2′OMe-microRNA-145.

Mature microRNAs (also referred to as miRNAs) are short, highly conserved, endogenous non-coding regulatory RNAs (18 to 24 nucleotides in length), expressed from longer, precursor transcripts (termed herein “pre-microRNAs” or “precursor microRNAs”) encoded in animal, plant and virus genomes, as well as in single-celled eukaryotes. Endogenous miRNAs found in genomes regulate the expression of target genes by binding to complementary sites, known as microRNA target sequences, in the mRNA transcripts of target genes to cause translational repression and/or transcript degradation. miRNAs have been implicated in processes and pathways such as development, cell proliferation, apoptosis, metabolism and morphogenesis, and in diseases including cancer (S. Griffiths-Jones et al., “miRBase: tools for microRNA genomics.” Nuc. Acid. Res., 2007: 36, D154-D158).

MicroRNA-145 is a putative tumor-suppressive microRNA that has been shown to be underexpressed in several types of tumors and causes cell growth inhibition by targeting c-Myc and IRS-1. Further, it has been described that microRNA-145 is able to target the pluripotency factors OCT4, SOX2, and KLF4 and functions as a key regulator of human stem cells (N. Xu et al., Cell 2009, 137:647-658), and promotes differentiation and represses proliferation of smooth muscle cells. MicroRNA-145 has also been reported to play an important role in p53-mediated repression of c-Myc. MicroRNA-145 has also been discussed as suppressing cell invasion and metastasis by directly targeting Mucin-1 (M. Sachdeva and Y-Y. Mo, Cancer Res, 2010 70(1):378-387).

Accordingly, as used herein, microRNA-145 refers to the mature microRNA having the human RNA sequence of: GUCCAGUUUUCCCAGGAAUCCCU (SEQ ID NO:1) together with any naturally occurring allelic, and further processed forms thereof. The pre-microRNA sequence of microRNA-145 refers to the human RNA sequence of ACCUUGUCCUCACGGUCCAGUUUUCCCAGGAAUCCCUUAGAUGCUAAGAUGGGGAUUCCU GGAAAUACUGUUCUUGAGGUCAUGGUU (SEQ ID NO:2), together with any naturally occurring allelic, variants, and processed forms thereof.

Inhibitory agents specific for microRNA-145, such as a microRNA-145 antagonist or inhibitor, for use in the cell-growth compositions and methods described herein, refer to any agent or small molecule (e.g., a compound) that inhibits, reduces, or decreases microRNA-145 activity or expression. Accordingly, as used herein the term “a microRNA-145 antagonist” refers to an agent that inhibits expression of a microRNA-145 polynucleotide or binds to, partially or totally blocks stimulation, decreases, prevents, delays activation, inactivates, desensitizes, or down regulates the activity of the microRNA-145 polynucleotide. Such inhibitors include agents that, e.g., inhibit expression or stability of a microRNA-145, or bind to, partially or totally block stimulation or any activity mediated by a microRNA-145 polynucleotide. Such inhibitors or antagonists include, but are not limited to, naturally occurring and synthetic ligands, antagonists, small molecules or compounds, antibodies, inhibitory nucleic acid sequence, inhibitory RNA molecules (i.e., siRNA or antisense RNA) and the like.

As used herein, an “RNA interference molecule” is defined as any agent which interferes with or inhibits expression of a target gene or genomic sequence, such as microRNA-145 polynucleotide having the sequence of SEQ ID NO:1 or SEQ ID NO:2, by RNA interference (RNAi). Such RNA interfering agents include, but are not limited to, nucleic acid molecules including RNA molecules which are homologous to the target gene or genomic sequence, or a fragment thereof, short interfering RNA (siRNA), short hairpin or small hairpin RNA (shRNA), microRNA (miRNA) and other nucleic acids that interfere with or inhibit expression of the target microRNA-145 polynucleotide gene by RNA interference (RNAi).

RNA interference (RNAi) refers to an evolutionally conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see Coburn, G. and Cullen, B., J. of Virology 76(18):9225 (2002)), thereby inhibiting expression of the target gene. In one embodiment, the RNA is double stranded RNA (dsRNA). In nature, RNAi is initiated by the dsRNA-specific endonuclease Dicer, which promotes processive cleavage of long dsRNA into double-stranded fragments termed siRNAs. siRNAs are incorporated into a protein complex (termed “RNA induced silencing complex,” or “RISC”) that recognizes and cleaves target mRNAs. RNAi can also be initiated by introducing nucleic acid molecules, e.g., synthetic siRNAs or RNA interfering agents, to inhibit or silence the expression of the target gene, microRNA-145, or a variant or precursor thereof. As used herein, “inhibition of microRNA-145” includes any decrease in expression level and/or activity of the microRNA-145, as compared to a situation wherein no RNA interference has been induced. The decrease can be of at least 20%, 30%, 40%, 50%, 60%, 70%, 80%, 90%, 95%, 99%, or more as compared to the expression of the microRNA-145, or the activity of the microRNA-145 that has not been targeted by an RNA interfering agent.

“Short interfering RNA” (siRNA), also referred to herein as “small interfering RNA” is defined as a nucleic acid-comprising agent that functions to inhibit expression of a target gene, by RNAi. An siRNA specific for or that targets a microRNA-145 or precursor thereof can be chemically synthesized, can be produced by in vitro transcription, or can be produced within a host cell. In some embodiments of the aspects described herein, an siRNA that targets a microRNA-145 is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, 22, or 23 nucleotides in length, and can contain a 3′ and/or 5′ overhang on each strand having a length of about 0, 1, 2, 3, 4, or 5 nucleotides. The length of the overhang is independent between the two strands, e.g., the length of the overhang on one strand is not dependent on the length of the overhang on the second strand. Preferably the siRNA is capable of promoting RNA interference through degradation or specific post-transcriptional gene silencing (PTGS) of the target microRNA-145.

siRNAs also include small hairpin (also called stem loop) RNAs (shRNAs). In one embodiment of the aspects described herein, an shRNA that targets a microRNA-145 is composed of or comprise a short (e.g., about 19 to about 25 nucleotide) antisense strand, followed by a nucleotide loop of about 5 to about 9 nucleotides, and the analogous sense strand. Alternatively, the sense strand can precede the nucleotide loop structure and the antisense strand can follow. These shRNAs can be encoded by plasmids, retroviruses, and lentiviruses and expressed from, for example, the pol III U6 promoter, or another promoter (see, e.g., Stewart, et al., RNA April; 9(4):493-501 (2003), incorporated by reference herein in its entirety).

The target gene or sequence of the RNA interfering agent can be a cellular gene or genomic sequence, e.g. the sequence encoding microRNA-145. An siRNA that targets a microRNA-145 can be substantially homologous to the target microRNA-145, or a fragment thereof. As used in this context, the term “homologous” is defined as being substantially identical, sufficiently complementary, or similar to the target RNA, or a fragment thereof, to effect RNA interference of the target. In addition to native RNA molecules, RNA suitable for inhibiting or interfering with the expression of a target sequence include RNA derivatives and analogs. Preferably, in some embodiments of the aspects described herein, the siRNA is identical in sequence to its target, i.e., the sequence encoding microRNA-145.

An siRNA specific for microRNA-145 preferably targets only micro-RNA 145. Any RNA interfering agent, such as an siRNA, can be screened for potential off-target effects by, for example, expression profiling. Such methods are known to one skilled in the art and are described, for example, in Jackson et al., Nature Biotechnology 6:635-637 (2003). In addition to expression profiling, one can also screen the potential target sequences for similar sequences in the sequence databases to identify potential sequences which can have off-target effects. For example, according to Jackson et al. (Id.) 15 contiguous nucleotides, or even as few as 11 contiguous nucleotides, of sequence identity are sufficient to direct silencing of non-targeted transcripts. Therefore, one can initially screen the proposed siRNAs to avoid potential off-target silencing using sequence identity analysis by any known sequence comparison methods, such as BLAST.

siRNA molecules need not be limited to those molecules comprising only RNA, but, for example, in some embodiments of the aspects described herein, further encompass chemically modified nucleotides and non-nucleotides that effect RNA interference, and also include molecules where a ribose sugar molecule is substituted for another sugar molecule or a molecule which performs a similar function. Moreover, a non-natural linkage between nucleotide residues can be used, such as a phosphorothioate linkage. The RNA strand can be derivatized with a reactive functional group or a reporter group, such as a fluorophore. Particularly useful derivatives are modified at a terminus or termini of an RNA strand, typically the 3′ terminus of the sense strand. For example, the 2′-hydroxyl at the 3′ terminus can be readily and selectively derivatized with a variety of groups.

Other useful RNA derivatives incorporate nucleotides having modified carbohydrate moieties, such as 2′O-alkylated residues or 2′-O-methyl ribosyl derivatives and 2′-O-fluoro ribosyl derivatives. The RNA bases can also be modified. Any modified base useful for inhibiting or interfering with the expression of the target microRNA-14 sequence can be used. For example, halogenated bases, such as 5-bromouracil and 5-iodouracil can be incorporated. The bases can also be alkylated, for example, 7-methylguanosine can be incorporated in place of a guanosine residue. Non-natural bases that yield successful inhibition can also be incorporated.

The most preferred siRNA modifications for the aspects and embodiments described herein include 2′-deoxy-2′-fluorouridine or locked nucleic acid (LNA) nucleotides and RNA duplexes comprising either phosphodiester or varying numbers of phosphorothioate linkages. Such siRNAs specific for microRNA 145 comprising such LNA or 2′-O-methyl modifications are referred to herein as an anti-microRNA-145 LNA oligonucleotide or an anti-microRNA-145 2′OMe oligonucleotide, respectively. Such modifications are known to one skilled in the art and are described, for example, in Braasch et al., Biochemistry 42: 7967-7975 (2003). Most of the useful modifications to the siRNA molecules can be introduced using chemistries established for antisense oligonucleotide technology. In some embodiments, the modifications involve minimal 2′-O-methyl modification, and in some embodiments, exclude such modifications. Modifications can also, in some embodiments, exclude modifications of the free 5′-hydroxyl groups of the siRNA.

siRNAs useful for targeting or inhibiting microRNA-145 expression can be readily designed and tested. Chalk et al. (Nucl. Acids Res. 33: D131-D134 (2005)) describe a database of siRNA sequences and a predictor of siRNA sequences. Linked to the sequences in the database is information such as siRNA thermodynamic properties and the potential for sequence-specific off-target effects. The database and associated predictive tools enable the user to evaluate an siRNA's potential for inhibition and non-specific effects. The database is available at on the world wide web at siRNA.cgb.ki.se.

Synthetic siRNA molecules, including shRNA molecules, can be obtained or produced using a number of techniques known to those of skill in the art. For example, the siRNA molecule can be chemically synthesized or recombinantly produced using methods known in the art, such as using appropriately protected ribonucleoside phosphoramidites and a conventional DNA/RNA synthesizer (see, e.g., Elbashir, S. M. et al., Nature 411:494-498 (2001); Elbashir, S. M., et al., Genes & Development 15:188-200 (2001); Harborth, J. et al., J. Cell Science 114:4557-4565 (2001); Masters, J. R. et al., Proc. Natl. Acad. Sci., USA 98:8012-8017 (2001); and Tuschl, T. et al., Genes & Development 13:3191-3197 (1999)). Alternatively, several commercial RNA synthesis suppliers are available including, but not limited to, Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). As such, siRNA molecules are not overly difficult to synthesize and are readily provided in a quality suitable for RNAi. In addition, dsRNAs can be expressed as stem loop structures encoded by plasmid vectors, retroviruses and lentiviruses (Paddison, P. J. et al., Genes Dev. 16:948-958 (2002); McManus, M. T. et al., RNA 8:842-850 (2002); Paul, C. P. et al., Nat. Biotechnol. 20:505-508 (2002); Miyagishi, M. et al., Nat. Biotechnol. 20:497-500 (2002); Sui, G. et al., Proc. Natl. Acad. Sci., USA 99:5515-5520 (2002); Brummelkamp, T. et al., Cancer Cell 2:243 (2002); Lee, N. S., et al., Nat. Biotechnol. 20:500-505 (2002); Yu, J. Y., et al., Proc. Natl. Acad. Sci., USA 99:6047-6052 (2002); Zeng, Y., et al., Mol. Cell 9:1327-1333 (2002); Rubinson, D. A., et al., Nat. Genet. 33:401-406 (2003); Stewart, S. A., et al., RNA 9:493-501 (2003)).

Accordingly, nucleic acid based micro-RNA 145 inhibitor agents for use in inhibition of microRNA-145 polynucloetides or precursors thereof include, but are not limited to, antisense oligomeric compounds, antisense oligonucleotides, siRNAs, alternate splicers, primers, probes and other compounds that hybridize to at least a portion of the target microRNA-145 nucleic acid or precursor thereof. Such nucleic acid based compounds are routinely prepared linearly, but can be joined or otherwise prepared to be circular and can also include branching. Separate nucleic acid based compounds can hybridize to form double stranded compounds that can be blunt-ended or can include overhangs on one or both termini. In general, a nucleic acid based micro-RNA 145 inhibitor agent comprises a backbone of linked monomeric subunits where each linked monomeric subunit is directly or indirectly attached to a heterocyclic base moiety. The linkages joining the monomeric subunits, the sugar moieties or sugar surrogates and the heterocyclic base moieties can be independently modified giving rise to a plurality of motifs for the resulting nucleic acid based micro-RNA 145 inhibitor agents, including hemimers, gapmers and chimeras.

### TGF-β Signaling Pathway

In some embodiments of the aspects described herein, the compositions and methods for generating a ChiPS cell comprise one or more activating agents specific for TGF-β signaling, such as a TGF-β signaling pathway agonist. In such embodiments, an activating agent specific for TGF-β signaling is a TGF-β polypeptide or an active fragment thereof, a fusion protein comprising a TGF-β polypeptide or an active fragment thereof, an agonist antibody to a TGF-β receptor, or a small molecule agonist of a TGF-β receptor.

The Transforming growth factor beta (TGF-β) signaling pathway is involved in many cellular processes in both the adult organism and the developing embryo including cell growth, cell differentiation, apoptosis, cellular homeostasis and other cellular functions. In spite of the wide range of cellular processes that the TGF-β signaling pathway regulates, the signaling mechanisms are relatively simple. TGF-β superfamily ligands bind to a type II receptor, which recruits and phosphorylates a type I receptor. The type I receptor then phosphorylates receptor-regulated SMADs (R-SMADs) which can now bind the coSMAD SMAD4. R-SMAD/coSMAD complexes accumulate in the nucleus where they act as transcription factors and participate in the regulation of target gene expression. TGF-β can be found in many different tissue types, including brain, heart, kidney, liver and testes.

TGF-β1 is a prototypic member of a family of cytokines including the TGF-βs, activins, inhibins, bone morphogenetic proteins and Mullerian-inhibiting substance, that signal through a family of single transmembrane serine/threonine kinase receptors. These receptors can be divided into two classes, the type I or activin like kinase (ALK) receptors and type H receptors. The ALK receptors are distinguished from the type II receptors in that the ALK receptors (a) lack the serine/threonine rich intracellular tail, (b) possess serine/threonine kinase domains that are very homologous between type I receptors, and (c) share a common sequence motif called the GS domain, consisting of a region rich in glycine and serine residues. The G_{S} domain is at the amino terminal end of the intracellular kinase domain and is critical for activation by the type II receptor. Several studies have shown that TGF-β signaling requires both the ALK and type H receptors. Specifically, the type II receptor phosphorylates the GS domain of the type I receptor for TGF-β, ALK5, in the presence of TGF-β. The ALK5, in turn, phosphorylates the cytoplasmic proteins Smad2 and Smad3 at two carboxy terminal serines. The phosphorylated Smad proteins translocate into the nucleus and activate genes that contribute to e.g., the production of extracellular matrix.

Activin ligands transduce signals in a manner similar to TGF-β ligands. Activins bind to and activate ALK receptors, which in turn phosphorylate Smad proteins such as Smad2 and Smad3. The consequent formation of a hetero-Smad complex with Smad4 results in the activin-induced regulation of gene transcription.

Smad proteins are exemplary downstream signal transduction factors in the TGF-beta pathway and therefore, in some embodiments, can be activated directly to effect reprogramming (e.g., by treating a cell with an activator of a Smad protein).

TGF-β receptors are single pass serine/threonine kinase receptors. They exist in several different isoforms that can be homo- or heterodimeric. The number of characterized ligands in the TGFβ superfamily far exceeds the number of known receptors, indicating that promiscuity exists between ligand and receptor interactions.

Three TGF-β receptor types can be distinguished by their structural and functional properties. Receptor types I and II have similar ligand binding affinities and can only be distinguished from each other by peptide mapping, both receptor types I and II have a high affinity for TGF-β1 and low affinity for TGF-β2. TGF-β receptor type III has a high affinity for both TGF-β 1 and -β2 and in addition TGF-β1.2.

Transforming growth factor, beta receptor I (herein termed “TGFBR1”) (activin A receptor type II-like kinase, 53 kDa) is a TGF beta receptor. TGFBR1 is its human gene. The protein encoded by this gene forms a heteromeric complex with type II TGF-beta receptors when bound to TGF-beta, transducing the TGF-beta signal from the cell surface to the cytoplasm. The encoded protein is a serine/threonine protein kinase. Mutations in this gene have been associated with Loeys-Dietz aortic aneurysm syndrome (LDAS).

Transforming growth factor, beta receptor II (70/80 kDa) is a TGF beta receptor. TGFBR2 is its human gene. This gene encodes a member of the Ser/Thr protein kinase family and the TGFB receptor subfamily. The encoded protein is a transmembrane protein that has a protein kinase domain, forms a heterodimeric complex with another receptor protein, and binds TGF-beta. This receptor/ligand complex phosphorylates proteins, which then enter the nucleus and regulate the transcription of a subset of genes related to cell proliferation. Mutations in this gene have been associated with Marfan Syndrome, Loeys-Deitz Aortic Aneurysm Syndrome, Osler-Weber-Rendu syndrome, and the development of various types of tumors. Alternatively spliced transcript variants encoding different isoforms have been characterized.

The TGF-β receptors contemplated for use in the methods described herein can be any TGF-β receptor including those from the Activin-like kinase family (ALK), the Bone Morphogenic Protein (BMP) family, the Nodal family, the Growth and Differentiation Factors family (GDF), and the TGF-β receptor family of receptors. TGF-β receptors are serine/threonine kinase receptors that effect various growth and differentiation pathways in the cell.

In some embodiments, a TGF-β receptor to be targeted using the cell-growth compositions and methods described herein is an ALK4, ALK5, or ALK7 receptor. In other embodiments, downstream effectors of any of the aforementioned TGF-beta receptor signaling pathways can be targeted, i.e., activated, directly to effect cell reprogramming with the cell growth compositions and methods described herein.

If desired, one of skill in the art can locate the protein sequence of any of the TGF-β receptors by simply searching “transforming growth factor beta receptor” in a protein sequence database such as NCBI. Some non-limiting examples of protein sequence accession numbers for TGF-β receptors are P36897.1 (SEQ ID NO: 3), Q5T7S2 (SEQ ID NO: 4), Q6IR47, P37173 (SEQ ID NO: 5), Q6A176, Q706C0, Q706C1, and Q03167.2 (SEQ ID NO: 6), among others.

Accordingly, provided herein are activating agents specific for TGF-β signaling, such as a TGF-β signaling pathway agonist for use in the cell-growth compositions and methods described herein.

As used herein, the terms “activating agent specific for TGF-β signaling” or “TGFβR activator” or “TGFβR agonist” refer to any agent or small molecule (e.g., a chemical compound) that activates, promotes, or increases TGF-β signal transduction by activating, promoting, or increasing production or expression of any of the factors constituting the TGF-β signal transduction system pathway, such as TGF-β ligand, TGF-β Type I receptors, TGF-β Type II receptors, TGF-β Type III receptors (β-glycan and endoglin), soluble forms of the TGF-β receptors, or Smad proteins. A TGF-β signaling pathway agonist refers to any agent, including small molecules, antibodies or antigen-binding fragments thereof against or specific for receptors and ligands implicated in the TGF-β signaling pathway, nucleic acid based molecules targeting pathway members, or any combination thereof, that activates the TGF-β signaling pathway.

The term “TGF-β signaling pathway” is used to describe the downstream signaling events attributed to TGF-β and TGF-β like ligands. For example, in one such signaling pathway, a TGF-β ligand binds to and activates a Type II TGF-β receptor. The Type II TGF-β receptor recruits and forms a heterodimer with a Type I TGF-β receptor. The resulting heterodimer permits phosphorylation of the Type I receptor, which in turn phosphorylates and activates a member of the SMAD family of proteins. A signaling cascade is triggered, and ultimately leads to control of the expression of mediators involved in cell growth, cell differentiation, tumorigenesis, apoptosis, and cellular homeostasis, among others. Other TGF-β signaling pathways are also contemplated for modulation according to the cell-growth compositions and methods described herein.

An activator or agonist of a TGFβR can function in a competitive or non-competitive manner. A TGFβR agonist includes any chemical or biological entity that, upon treatment of a cell, results in an increase in a biological activity caused by activation of the TGFβR in response to binding of its natural ligand.

While any TGF-β signaling pathway agonist can potentially be used in the methods described herein, it is preferable that a TGF-β signaling pathway agonist is either selective for, or specific for, a member of the TGF-β signaling pathway. By “specific” is meant that at the dose necessary for the activating agent to activate the TGF-β signaling pathway, the activating agent does not have any other substantial pharmacological action in the cell or host. By “selective” is meant that the dose of the activating agent necessary for activation of the TGF-β signaling pathway is at least 2-fold lower than the dose necessary for activation or inhibition of another pharmacological action as measured by the ED₅₀ or EC₅₀ of the agent for each pharmacological effect; preferably the dose of activating agent necessary for TGF-β pathway activation is at least 5-fold lower, at least 10 fold lower, at least 20-fold lower, at least 30-fold lower, at least 40-fold lower, at least 50-fold lower, at least 60-fold lower, at least 70-fold lower, at least 80-fold lower, at least 90-fold lower, at least 100-fold lower, at least 500-fold lower, at least 1000 fold lower or more, than the dose necessary for another pharmacological action. Thus, to be clear, the agents useful for the methods described herein primarily activate the TGF-β signaling pathway with only minor, if any, effects on other pharmacological pathways, and the dose used for activation of the TGF-β signaling pathway is sub-clinical or sub-threshold for other pharmacological responses.

Such an agonist can act by binding to the extracellular domain of a TGF-β receptor and by initiating its serine/threonine kinase activity (e.g., ATP binding site). In addition, the TGFβR inhibitor can also bind to a non-ligand binding site and, for example, produce a conformational shift in the TGFβR, such that binding of the endogenous ligand of the TGFβR is promoted. The receptor activity of a TGF-β receptor can be measured, for example, as described by Laping, N J., et al (2002) Molecular Pharmacology 62(1):58-64, which is herein incorporated by reference in its entirety.

An agent is determined to be a TGF-β signaling pathway agonist if the level of phosphorylation of the Type I TGF-β receptor in a culture of cells is increased by at least 20% compared to the level of phosphorylation of the Type I TGF-β receptor in cells that are cultured in the absence of a TGF-β signaling pathway agonist; preferably the level of phosphorylation is increased by at least 30%, at least 40%, at least 50%, at least 60%, at least 70%, at least 80%, at least 90%, at least 95%, at least 99%, or even 100%, at least two-fold, at least five-fold, at least 10-fold, at least 25-fold, or greater, in the presence of a TGF-β signaling pathway agonist. In addition, the dose-response curve for a TGF-β receptor agonist can be determined by measuring TGF-β receptor activity over a variety of agonist concentrations using the method of Laping, N J., et al (2002).

Non-limiting examples of TGF-β signaling pathway agonists for use in the cell-growth compositions and methods described herein include a TGF-β polypeptide or an active fragment thereof (recombinantly generated, or obtained from a natural source), a fusion protein comprising a TGF-β polypeptide or an active fragment thereof, an agonist antibody or antigen-binding fragment thereof specific for any of the TGF-β receptors, whereby binding of the antibody or antigen-binding fragment to the TGF-β receptor activates the receptor, or mimics the natural activity of TGF-β binding to the receptor, or a small molecule agonist of a TGF-β receptor. Recombinant TGF-β polypeptide and active fragments thereof can be obtained from any commercial source providing the same.

Other non-limiting examples of TGF-β signaling pathway agonists for use in the cell-growth compositions and methods described herein include agents that directly stimulate TGF-β production. Such agents, referred to herein as “TGF-β production stimulators,” include, but are not limited to, triphenylethylene (TPE) derivatives, such as Tamoxifen, and any of the triphenylethylene compounds described in US Patent Publication 20100099642, the contents of which are herein incorporated by reference in their entireties.

### Other Molecules to Increase Efficiency of Reprogramming of Differentiated Cells

In some embodiments of the aspects described herein, the efficiency of reprogramming (e.g., the number of reprogrammed cells) can be enhanced by the addition of various small molecules as shown by Shi, Y., et al (2008) Cell-Stem Cell 2:525-528, Huangfu, D., et al (2008) Nature Biotechnology 26(7):795-797, Marson, A., et al (2008) Cell-Stem Cell 3:132-135, which are incorporated herein by reference in their entirety. It is contemplated that the compositions and methods described herein can also be used in combination with an additional single small molecule (or any combination of such small molecules) that enhance(s) the efficiency of production of a reprogrammed ChiPS cell. Some non-limiting examples of agents that enhance reprogramming efficiency include soluble Wnt, Wnt conditioned media, BIX-01294 (a G9a histone methyltransferase), PD0325901 (a MEK inhibitor), DNA methyltransferase inhibitors, histone deacetylase (HDAC) inhibitors, valproic acid, 5′-azacytidine, dexamethasone, suberoylanilide, hydroxamic acid (SAHA), and trichostatin (TSA), among others. Thus, it is contemplated herein that inhibitors of microRNA-145 and activators of the TGF-β signaling pathway described herein can be used either alone or in combination with another small molecule (or any combination of small molecules) to enhance or increase the efficiency of producing reprogrammed cells from somatic cells as disclosed herein. Any agent that increases efficiency of production of reprogrammed cells for use with the methods and compositions described herein are referred to as a “reprogramming enhancing agent”.

A reprogramming enhancing agent can increase the efficiency of production of reprogrammed cells or increase the rate of production of reprogrammed cells. By “increasing the efficiency” of reprogrammed cell production is meant that the percentage of reprogrammed cells in a given population of cells is at least 5% higher in populations treated with a such an agent (e.g. reprogramming enhancing agent) than a comparable, control treated population, i.e., population of cells not treated or administered the reprogramming enhancing agent. It is preferred that the percentage of reprogrammed cells in a reprogramming enhancing agent-treated population is at least 10% higher, at least 20% higher, at least 30% higher, at least 40% higher, at least 50% higher, at least 60% higher, at least 70% higher, at least 80% higher, at least 90% higher, at least 1-fold higher, at least 2-fold higher, at least 5-fold higher, at least 10 fold higher, at least 100 fold higher, at least 1000-fold higher or more than a control treated population of comparable size and culture conditions. The term “control treated population of comparable size and culture conditions” is used herein to describe a population of cells that has been treated with identical media, viral induction, nucleic acid sequence, temperature, confluency, flask size, pH, etc., with the exception (e.g. absence) of the reprogramming enhancing agent. To be clear, the only difference between a control treated population and a reprogramming enhancing agent-treated cell population is the condition of having been treated with a reprogramming enhancing agent.

By “increasing the rate” of production of reprogrammed cells is meant that the amount of time for the induction of chemical human induced pluripotent stem cells, is at least 2 days less than in a control treated population of comparable size and culture conditions in the absence of the reprogramming enhancing agent; preferably the time needed for chemical human induced pluripotent stem cell induction is at least 3 days less, at least 4 days less, at least 5 days less, at least 6 days less, at least 1 week less, at least 2 weeks less, at least 3 weeks less or more, in the presence of the reprogramming enhancing agent than in a control treated population.

Accordingly, in some embodiments, the somatic cell is further contacted with a HDAC inhibitor, e.g., a HDAC inhibitor described herein or an inhibitor of DNA methyltransferase, e.g., a DNA methyltransferase inhibitor described herein. In some embodiments, the HDAC inhibitor is one or more of valproic acid (VPA), suberoylanilide hydroxamic acid (SAHA) and trichstatin A (TSA). In one embodiment, the methods include contacting a differentiated cell with VPA. In one embodiment, the DNA methyltransferase inhibitor is 5-aza-Cytidine (5azaC).

Other non-limiting examples of reprogramming enhancing agents include: Suberoylanilide Hydroxamic Acid (SAHA (e.g., MK0683, vorinostat) and other hydroxamic acids), BML-210, Depudecin (e.g., (−)-Depudecin), HC Toxin, Nullscript (4-(1,3-Dioxo-1H,3H-benzo[de]isoquinolin-2-yl)-N-hydroxybutanamide), Phenylbutyrate (e.g., sodium phenylbutyrate) and Valproic Acid ((VPA) and other short chain fatty acids), Scriptaid, Suramin Sodium, Trichostatin A (TSA), APHA Compound 8, Apicidin, Sodium Butyrate, pivaloyloxymethyl butyrate (Pivanex, AN-9), Trapoxin B, Chlamydocin, Depsipeptide (also known as FR901228 or FK228), benzamides (e.g., CI-994 (e.g., N-acetyl dinaline) and MS-27-275), MGCD0103, NVP-LAQ-824, CBHA (m-carboxycinnaminic acid bishydroxamic acid), JNJ16241199, Tubacin, A-161906, proxamide, oxamflatin, 3-Cl-UCHA (e.g., 6-(3-chlorophenylureido)caproic hydroxamic acid), AOE (2-amino-8-oxo-9,10-epoxydecanoic acid), CHAP31 and CHAP 50. Other reprogramming enhancing agents include, for example, dominant negative forms of the HDACs (e.g., catalytically inactive forms), siRNA inhibitors of the HDACs, and antibodies that specifically bind to the HDACs. Such inhibitors are available, e.g., from BIOMOL International, Fukasawa, Merck Biosciences, Novartis, Gloucester Pharmaceuticals, Aton Pharma, Titan Pharmaceuticals, Schering AG, Pharmion, MethylGene, and Sigma Aldrich. In some embodiments, VPA is a preferred histone deacetylase inhibitor.

### Confirming Cellular Reprogramming

In some aspects, the ChiPS cells produce one or more markers indicative of a pluripotent stem cell, such as an iPS cell generated using any of the previously described methods. In some embodiments, the methods can include detecting a marker indicative of a reprogrammed cells. In some embodiments, the marker can be detected using a reagent, e.g., a reagent for the detection of alkaline phosphatase (AP), NANOG, OCT-4, SOX-2, SSEA4, TRA-1-60 or TRA-1-81, e.g., an antibody against the marker or primers for a RT-PCR or PCR reaction, e.g., a semi-quantitative or quantitative RT-PCR or PCR reaction. Such markers can be used to evaluate whether a reprogrammed cell (e.g. an ChiPS cell) has been produced.

In other embodiments, the expression of alkaline phosphatase (AP), NANOG, OCT-4, SOX-2, SSEA4, TRA-1-60 or TRA-1-81 in a reprogrammed cell or population of cells, i.e., ChiPS cells, is at least 50% higher, at least 60% higher, at least 70% higher, at least 80% higher, at least 90% higher, at least about 1-fold higher, is at least about 2-fold higher, at least about 3-fold higher, at least about 4-fold higher, at least about 6-fold higher, at least about 8-fold higher, at least about 10-fold higher, at least about 15-fold higher, at least about 20-fold higher, at least about 40-fold higher, at least about 80-fold higher, at least about 100-fold higher, at least about 150-fold higher, at least about 200-fold higher, at least about 500-fold higher, at least about 750-fold higher, at least about 1000-fold higher, at least about 2500-fold higher, at least about 5000-fold higher, at least about 7500-fold higher or at least about 10,000-fold higher than the expression of alkaline phosphatase (AP), NANOG, OCT-4, SOX-2, SSEA4, TRA-1-60 or TRA-1-81 in the somatic cell or cells from which the reprogrammed cell or population of cells was derived or generated from. In some embodiments, the method to determine the presence of a reprogrammed cell further comprises performing an analysis of the karyotype of the ChiPS cell.

In some embodiments, the presence of a reprogrammed ChiPS cell in a pluripotent state produced by the methods as disclosed herein is determined using methods which compare the chemically induced reprogrammed cells (ChiPS cells) with ES cells in terms of morphology, proliferation, gene expression, and teratoma formation, according to methods as disclosed herein in the Examples. One can also assess if the reprogrammed cells are in a pluripotent state by assessing if they give rise to adult chimeras which are competent for germline transmission when transplanted into blastocysts, as disclosed in Maherali et al., Cell Stem Cell 1:55-70, 2007; Okita et al., Nature 448:313-17, 2007; Wemig et al., Nature 448:318-324, 2007, which are incorporated herein in their entirety by reference. Additionally, one of ordinary skill in the art can also assess the ChiPS cells to differentiate into cell types of the three germ layers in vitro and in teratomas, as shown herein in the Examples.

In some embodiments, the ability of the reprogrammed ChiPS cell to form a teratomas, or to differentiated into all three germ layers in vitro is at least about 4-fold higher, at least about 6-fold higher, at least about 8-fold higher, at least about 10-fold higher, at least about 15-fold higher, at least about 20-fold higher, at least about 40-fold higher, at least about 80-fold higher, at least about 100-fold higher, at least about 150-fold higher, at least about 200-fold higher or more than 200-fold higher as compared to the ability of the somatic cell from which the reprogrammed cell was derived to form a teratomas, or to differentiated into all three germ layers in vitro.

The reprogrammed ChiPS cells as disclosed herein can express any number of pluripotent cell markers, including: alkaline phosphatase (AP); ABCG2; stage specific embryonic antigen-1 (SSEA-1); SSEA-3; SSEA-4; TRA-1-60; TRA-1-81; Tra-2-49/6E; ERas/ECAT5, E-cadherin; .beta.III-tubulin; .alpha.-smooth muscle actin (.alpha.-SMA); fibroblast growth factor 4 (Fgf4), Cripto, Dax1; zinc finger protein 296 (Zfp296); N-acetyltransferase-1 (Nat1); (ES cell associated transcript 1 (ECAT1); ESG1/DPPA5/ECAT2; ECAT3; ECAT6; ECAT7; ECAT8; ECAT9; ECAT10; ECAT15-1; ECAT15-2; Fthl17; Sal14; undifferentiated embryonic cell transcription factor (Utf1); Rex1; p53; G3PDH; telomerase, including TERT; silent X chromosome genes; Dnmt3a; Dnmt3b; TRIM28; F-box containing protein 15 (Fbx15); Nanog/ECAT4; Oct3/4; Sox2; Klf4; c-Myc; Esrrb; TDGF1; GABRB3; Zfp42, FoxD3; GDF3; CYP25A1; developmental pluripotency-associated 2 (DPPA2); T-cell lymphoma breakpoint 1 (Tcl1); DPPA3/Stella; DPPA4; other general markers for pluripotency, etc. Other markers can include Dnmt3L; Sox15; Stat3; Grb2; SV40 Large T Antigen; HPV16 E6; HPV16 E7, β-catenin, and Bmi1. Such cells can also be characterized by the down-regulation of markers characteristic of the somatic cell from which the ChiPS cell is induced.

Enrichment, Isolation and/or Purification of Reprogrammed ChiPS Cells

Other embodiments of the methods described herein relate to the isolation of a population of reprogrammed ChiPS cells from a heterogeneous population of cells, such a mixed population of cells comprising reprogrammed ChiPS cells and differentiated cells from which the reprogrammed ChiPS cells were derived. A population of reprogrammed cells produced by any of the above-described processes can be enriched, isolated and/or purified by using any cell surface marker present on the reprogrammed cell which is not present on the differentiated cell from which it was derived. Such cell surface markers are also referred to as an affinity tag which is specific for reprogrammed cells.

Using the methods described herein, enriched for, isolated and/or purified populations of reprogrammed ChiPS cells can be produced in vitro from somatic cells, which have undergone sufficient reprogramming to produce at least some reprogrammed ChiPS cells. In a preferred method, the somatic cells are reprogrammed primarily into ChiPS cells. Some preferred enrichment, isolation and/or purification methods relate to the in vitro production of reprogrammed ChiPS cells from human somatic cells (such as fibroblasts).

Using the methods described herein, isolated cell populations of reprogrammed ChiPS cells are enriched in reprogrammed content by at least about 2- to about 1000-fold as compared to a population before reprogramming of the somatic cells. In some embodiments, reprogrammed ChiPS cells can be enriched by at least about 5- to about 500-fold as compared to a population before reprogramming of the somatic cells. In other embodiments, reprogrammed cells can be enriched from at least about 10- to about 200-fold as compared to a population before reprogramming of the differentiated cells. In still other embodiments, reprogrammed cells can be enriched from at least about 20- to about 100-fold as compared to a population before reprogramming of the differentiated cells. In yet other embodiments, reprogrammed ChiPS cells can be enriched from at least about 40- to about 80-fold as compared to a population before reprogramming of the somatic cells. In certain embodiments, reprogrammed ChiPS cells can be enriched from at least about 2- to about 20-fold as compared to a population before reprogramming of the somatic cells.

The terms “isolate” and “methods of isolation,” as used herein, refer to any process whereby a cell or population of cells is removed from a subject or sample in which it was originally found, or a descendant of such a cell or cells. The term “isolated population,” as used herein, refers to a population of cells that has been removed and separated from a biological sample, or a mixed or heterogeneous population of cells found in such a sample. In some embodiments, an isolated cell or cell population is further cultured in vitro or ex vivo, to further expand, for example, the number of reprogrammed cells. Such cultures can be performed using any method known to one of skill in the art, for example, as described in the Examples section.

The term “substantially pure” or “purified” with respect to a particular cell population, refers to a population of cells that is at least about 75%, at least about 80%, at least about 85%, at least about 90%, at least about 95%, at least about 98%, or at least about 99% pure, with respect to the cells making up a total cell population. Some embodiments of the aspects described herein further encompass methods to expand a population of reprogrammed somatic cells, wherein the expanded population of reprogrammed somatic cells is a substantially pure or enriched population of reprogrammed somatic cells.

The terms “enriching for or “enriched for” are used interchangeably herein and mean that the yield (fraction) of cells of one type, such as reprogrammed somatic cells produced using the compositions and methods described herein, is increased by at least 15%, by at least 20%, by at least 25%, by at least 30%, by at least 35%, by at least 40%, by at least 45%, by at least 50%, by at least 55%, by at least 60%, by at least 65%, by at least 70%, or by at least 75%, over the fraction of cells of that type in the starting biological sample, culture, or preparation. Methods to isolate a substantially pure or enriched population of reprogrammed somatic cells available to a skilled artisan include, but are not limited to, immunoselection techniques, such as high-throughput cell sorting using flow cytometric methods, affinity methods with antibodies labeled to magnetic beads, biodegradable beads, non-biodegradable beads, and antibodies panned to surfaces including dishes, and any combination of such methods, as well as separation based on other physical properties (Doevendans et al. (2000) J. Mol. Cell. Cardiol. 32:839-851). Other means of positive selection include drug selection, for instance such as described by Klug et al., involving enrichment of desired cells by density gradient centrifugation. Negative selection can be performed and selecting and removing cells with undesired markers or characteristics, for example fibroblast markers, epithelial cell markers etc.

### Kits

Other aspects described herein relate to kits for use in practicing the methods for making the reprogrammed ChiPS cells described herein.

Accordingly, in one aspect, a kit comprises: an inhibitor of microRNA-145, an activator or agonist of TGF-β signaling, and instructions and packaging thereof for converting a somatic cell to a reprogrammed ChiPS cell using any of the methods described herein.

In some embodiments, the agents and components described herein, such as a small molecule inhibitor of microRNA-145 or a nucleic acid inhibitor of microRNA-145 (e.g., a nucleic acid inhibitor of microRNA-145 can be a anti-microRNA-145 LNA oligonucleotide or a 2′OMe-microRNA-145), or the activator of TGF-β (TGF-(or active fragment thereof) can be provided singularly or in any combination as a kit. The kit includes (a) the compounds described herein, e.g., a composition(s) that includes a compound(s) described herein, and, optionally (b) informational material.

It is preferred that any of the modulating agent(s) described herein (i.e., an inhibitor of microRNA-145, an activator or agonist of TGF-(signaling) for use in the kits be substantially pure and/or sterile. When an agent (s) described herein is provided in a liquid solution, the liquid solution preferably is an aqueous solution, with a sterile aqueous solution being preferred. When an agent (s) described herein is provided as a dried form, reconstitution generally is by the addition of a suitable solvent. The solvent, e.g., sterile water or buffer, can optionally be provided in the kit.

The informational material can be descriptive, instructional, marketing or other material that relates to the methods described herein and/or the use of an agent(s) described herein for the methods described herein.

The informational material of the kits is not limited in its form. In one embodiment, the informational material can include information about production of the agent(s), molecular weight of the agent(s), concentration, date of expiration, batch or production site information, and so forth. In one embodiment, the informational material relates to methods for administering the agent(s).

In one embodiment, the informational material can include instructions to administer an agent(s) described herein in a suitable manner to perform the methods described herein, e.g., in a suitable dose, dosage form, or mode of administration (e.g., a dose, dosage form, or mode of administration described herein) (e.g., to a cell in vitro or a cell in vivo). In another embodiment, the informational material can include instructions to administer an agent(s) described herein to a suitable subject, e.g., a human, e.g., a human having or at risk for a disorder described herein or to a cell in vitro.

In addition to any of the agent(s) described herein, the composition of the kit can include other ingredients, such as a solvent or buffer, a stabilizer, a preservative, a flavoring agent (e.g., a bitter antagonist or a sweetener), a fragrance or other cosmetic ingredient, and/or an additional agent, e.g., for inducing reprogrammed ChiPS cells (e.g., in vitro) or for treating a condition or disorder described herein. Alternatively, the other ingredients can be included in the kit, but in different compositions or containers than a compound described herein. In such embodiments, the kit can include instructions for admixing an agent (s) described herein and the other ingredients, or for using an agent (s) described herein together with the other ingredients, e.g., instructions on combining the two agents prior to administration.

An agent(s) described herein can be provided in any form, e.g., liquid, dried or lyophilized form. It is preferred that an agent(s) described herein be substantially pure and/or sterile. When an agent(s) described herein is provided in a liquid solution, the liquid solution preferably is an aqueous solution, with a sterile aqueous solution being preferred. When an agent(s) described herein is provided as a dried form, reconstitution generally is by the addition of a suitable solvent. The solvent, e.g., sterile water or buffer, can optionally be provided in the kit.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In a preferred embodiment, the device is a medical implant device, e.g., packaged for surgical insertion.

The kit can further include a component for the detection of a marker for reprogrammed cells, e.g., for a marker described herein, e.g., a reagent for the detection of alkaline phosphatase (AP), NANOG, OCT-4, SOX-2, SSEA4, TRA-1-60 or TRA-1-81, e.g., an antibody against the marker or primers for a RT-PCR or PCR reaction, e.g., a semi-quantitative or quantitative RT-PCR or PCR reaction. Such markers can be used to evaluate whether an reprogrammed ChiPS cell has been produced. Accordingly, the kit can further comprises one or more components for karyotyping, e.g., a probe, a dye, a substrate, an enzyme, an antibody or other useful reagents for preparing a karyotype from a cell. The kit can include an iPS cell, e.g., an iPS cell derived from the same cell type as the somatic cell. In one embodiment, the iPS cell can be for use as a control. The kit can also further comprise an HDAC inhibitor(s), e.g. VPA. In some embodiments, the kit further comprises a DNA methyltransferase inhibitor (e.g., 5azaC).

### Assessing and Modulating p21 Expression to Prevent Cancerous Transformation

Provided herein are methods to assess and decrease the risk of cancerous transformation of an induced pluripotent stem cell generated using any method known to one of skill in the art, including the novel methods described herein of generating ChiPS cells. These methods are based on the novel discovery by the inventors that the ratio of expression of p21 to the expression of p53 provides a measurement of risk of cancerous transformation of an induced pluripotent stem cell.

Accordingly, in one aspect, methods of assessing the risk of cancerous transformation of an induced pluripotent stem (iPS) cell are provided. Such methods comprise measuring an expression of p21 and an expression of p53 in an induced pluripotent stem cell, and using the information provided by these measurements to determine a risk of cancerous transformation, such that an increased expression of p21 relative to the expression of p53 in the induced pluripotent stem cell is indicative of a decreased risk of cancerous transformation.

In such methods of assessing the risk of cancerous transformation of a cell or cell population, the method can comprise isolating, purifying, or enriching for one or more induced pluripotent stem cells, or a population of induced pluripotent stem cells, and measuring expression of p21 and p53 using any technique known to one of skill in the art. For example, in some embodiments, RNA can be isolated from the induced pluripotent stem cells, and expression of p21 and p53 can be determined using qRT-PCR, semi-quantitative PCR, or Northern blot analysis. In other embodiments, a cellular lysate can be isolated from the induced pluripotent stem cells, and protein expression of p21 and p53 can be determined using Western blot analyses, or by, for example, bead-based methods. In other embodiments, protein expression of p21 and p53 in the induced pluripotent stem cells, can be determined using flow-cytometric methods, following intracellular staining techniques. Having obtained these measurements, a ratio of p21 to p53 expression can be determined, such that when the ratio of p21:p53 expression is at least greater than 1, at least greater than 1.5, is at least greater than 2, at least greater than 3, at least greater than 4, at least greater than 5, is at least greater than 6, at least greater than 7, at least greater than 8, at least greater than 9, is at least greater than 10, at least greater than 25, at least greater than 50, at least greater than 100, or greater, than the induced pluripotent stem cell or population of induced pluripotent stem cells is said to have a decreased risk of cancerous transformation.

In another aspect, methods of decreasing the risk of cancerous transformation of an induced pluripotent stem (iPS) cell are provided. These methods comprise contacting an induced pluripotent stem cell with one or more agents that induce p21 expression in the induced pluripotent stem cell, such that the induction of p21 expression in the induced pluripotent stem cell decreases the risk of cancerous transformation. In some embodiments of these methods, agents that increase the ratio of p21 to p53 in a cell by targeting p53 expression and or p53 activity are also contemplated.

As used herein, “p21” refers to the cyclin-dependent kinase inhibitor (CKI). The p21 (also known as “WAF1”) protein binds to and inhibits the activity of cyclin-CDK2 or -CDK4 complexes, and thus functions as a regulator of cell cycle progression at G1. The expression of this gene is tightly controlled by the tumor suppressor protein p53, through which p21 protein mediates the p53-dependent cell cycle G1 phase arrest in response to a variety of stress stimuli. In addition to growth arrest, p21 can mediate cellular senescence, it was originally discovered as a senescent cell-derived inhibitor. The p21 (WAF1) protein can also interact with proliferating cell nuclear antigen (PCNA), a DNA polymerase accessory factor, and plays a regulatory role in S phase DNA replication and DNA damage repair. Two alternatively spliced variants, which encode an identical protein, have been reported.

p21 (WAF1) is a CKI that directly inhibits the activity of cyclin E/CDK2 and cyclin D/CDK4 complexes. p21 functions as a regulator of cell cycle progression at S phase. The expression of p21 is controlled by the tumor suppressor protein p53. Sometimes, it is expressed without being induced by p53. This kind of induction plays an important role in p53 independent differentiation which is promoted by p21. Expression of p21 is mainly dependent on two factors 1) stimulus provided 2) type of the cell. Growth arrest by p21 can promote cellular differentiation. Accordingly, in some embodiments of the methods described herein, p21 is induced independently of p53 expression or activity.

As used herein, p53 (also known as protein 53 or tumor protein 53) refers to a tumor suppressor protein that in humans is encoded by the TP53 gene. p53 is important in multicellular organisms, where it regulates the cell cycle and, thus, functions as a tumor suppressor that is involved in preventing cancer. As such, p53 has been described as the guardian of the genome,” the “guardian angel gene,” and the “master watchman,” referring to its role in conserving stability by preventing genome mutation.

Exemplary agents for use in inducing p21 expression and/or activity, or increasing the ratio of p21:p53 in an induced pluripotent stem cell according to the methods described herein include PRIMA-1 (2,2-Bis(hydroxymethyl)-1-azabicyclo[2.2.2]octan-3-one), which selectively restores mutant p53 activity in tumor cells via activation of Bax and PUMA, and induces apoptosis and inhibits growth of human tumors with mutant p53; arsenic trioxide (As₂O₃); Daunomycin; Apicidin; inhibitors of HDAC1 and HDAC2, such as 5-phenyl-2,4-pentadienoyl hydroxamic acid, N-methyl-5-phenyl-2,4-pentadienoyl hydroxamic acid, 3-methyl-5-phenyl-2,4-pentadienoyl hydroxamic acid, 4-methyl-5-phenyl-2,4-pentadienoyl hydroxamic acid, 4-chloro-5-phenyl-2,4-pentadienoyl hydroxamic acid, 5-(4-dimethylaminophenyl)-2,4-pentadienoyl hydroxamic acid, 5-phenyl-2-en-4-yn-pentanoyl hydroxamic acid, N-methyl-6-phenyl-3,5-hexadienoyl hydroxamic acid, potassium 2-oxo-6-phenyl-3,5-hexadienoate, potassium 2-oxo-8-phenyl-3,5,7-octatrienoate, 7-phenyl-2,4,6-hepta-trienoylhydroxamic acid, or derivatives thereof; cis-Diamminedichloroplatinum; or any agent or compound that inhibits microRNA-106(b) family members. In addition, any of the synthetic dsRNAs targeting p21 promoter regions that can induce gene expression in the phenomenon referred to as dsRNA-induced gene activation/RNA activation, as described in Mol Cancer Ther. 2008 March; 7(3):698-703, which is herein incorporated by reference in its entirety, are also contemplated as agents for use in inducing p21 expression and/or activity in the methods described herein.

### Redifferentiating and Hepatocyte-Like Cells

Also provided herein are compositions, methods, and kits for redifferentiating a cancerous hepatoma stem cell to a hepatocyte-like cell. These aspects are based on the novel discovery by the inventors that contacting a hepatoma cell with acyclic retinoid and an aldo-keto reductase family 1 B10 (AKR1B10) inhibitor causes redifferentiation of the hepatoma cell to a hepatocyte-like cell.

Hepatocellular carcinoma (HCC, also called malignant hepatoma) is a primary malignancy (i.e., cancer) of the liver. Most cases of HCC are secondary to either a viral hepatitide infection (hepatitis B or C) or cirrhosis (alcoholism being the most common cause of hepatic cirrhosis). In countries where hepatitis is not endemic, most malignant cancers in the liver are not primary HCC but metastasis (spread) of cancer from elsewhere in the body, e.g., the colon. Treatment options of HCC and prognosis are dependent on many factors but especially on tumor size and staging. Tumor grade is also important. High-grade tumors will have a poor prognosis, while low-grade tumors may go unnoticed for many years, as is the case in many other organs.

Hepatocellular carcinoma (HCC) is the most common type of adult liver cancer, and is the third leading cause of cancer deaths worldwide (Block T M et al. (2003), Oncogene 22, pp. 5093-5107). Many patients with HCC remain asymptomatic until the disease is in its advanced stages, resulting in ineffective treatment and poor prognosis; the majority of unresectable HCC patients die within one year. The clinical management of HCC can be expected to improve dramatically with improved screening tools to detect the carcinoma in the early stage.

The major risk factors of HCC are chronic infections with hepatitis B or hepatitis C virus (HBV or HCV, respectively). Chronic hepatitis can progress into cirrhosis (a noncancerous liver disease associated with fibrosis and abnormal nodules), which increases the risk of developing HCC. Patients with chronic hepatitis and/or cirrhosis, therefore, form a high risk population which would benefit from regular screening for HCC. Current screening tests for HCC are the measurement of alpha-fetoprotein (AFP) levels in the blood serum and the conduction of a hepatic ultrasound. Elevated serum AFP is, however, not a specific marker for HCC, since it is detected in a wide variety of non-hepatic malignancies and benign conditions, including acute and chronic hepatitis (McIntire K R et al. (1975), Cancer Res. 35, pp. 991-996; Liaw Y F (1986), Liver 6, pp. 133-137). Furthermore, 30-50% of HCC cases do not present with elevated serum AFP {Johnson P J (2001), Clin. Liver Dis. 5, pp. 145-159}. As a consequence, the AFP test can miss 50% of the positives due to its lack of sensitivity and specificity. A majority of HCC patients concomitantly suffers from cirrhosis. In those patients, the use of advanced imaging technology such as hepatic ultrasound is difficult and frequently non-conclusive.

The inventors have discovered that cancer cells from a hepatocellular carcinoma or hepatoma, in particular those cancer cells expressing CD133, can be induced to redifferentiate into a hepatocyte-like cell using the compositions and methods described herein, which comprise acyclic retinoid and an aldo-keto reductase family 1 B10 (AKR1B10) inhibitor.

Accordingly, in one aspect, cell-growth compositions for redifferentiating hepatoma cells are provided. Such cell-growth compositions comprise acyclic retinoid, and one or more aldo-keto reductase family 1 B10 (AKR1B10) inhibitors.

Thus, a cell-growth composition for redifferentiating hepatoma cells refers to a composition comprising one or more agents, such as acyclic retionoid and one or more inhibitors of AKR1B10, for modulating the growth and differentiation of a hepatoma cell to which the cell-growth composition is added or contacted with. Accordingly, in some embodiments, a hepatoma cell contacted with the cell-growth composition undergoes redifferentiation to a hepatocyte-like state. In some embodiments of the aspects described herein, a cell-growth composition can be added to or formulated with a cell culture medium for culturing cells that contains nutrients that maintain cell viability and support proliferation. Such a cell culture medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types, such as hepatoma cells, are known to those skilled in the art.

In others aspects, pharmaceutical compositions for redifferentiating hepatoma cells are provided. Such pharmaceutical compositions comprise acyclic retinoid, one or more aldo-keto reductase family 1 B10 (AKR1B10) inhibitors, and a pharmaceutically acceptable carrier. In some embodiments of such aspects, the pharmaceutical compositions for redifferentiating hepatoma cells are administered to a subject in need, i.e., a subject having or at risk for hepatocellular carcininoma.

In another aspect, methods for redifferentiating a hepatoma cell to a hepatocyte-like cell are provided. Such methods comprise contacting a cell a hepatoma cell with acyclic retinoid and an aldo-keto reductase family 1 B10 (AKR1B10) inhibitor, such that redifferentiation of the hepatoma cell occurs and a hepatocyte-like cell is generated. In some embodiments of these methods, the human hepatoma cell is positive for CD133. In some embodiments of these methods, the AKR1B10 inhibitor is tolrestat. In some embodiments of these aspects, a level of secretion of albumin, alpha fetoprotein, or a combination therein is measured before and after the contacting of the hepatoma cell.

As used herein, the term “redifferentiating” refers to a process that alters or changes the differentiation state of a cancer cell, such as a hepatoma cell, or a cancer stem cell, to a different differentiation state that is not associated with a cancerous state. Stated another way, redifferentiating, when used in reference to a cancer cell, refers to a process of changing the differentiation state of a cancer cell to another or different differentiation state, wherein the cancer cell is no longer cancerous. In preferred embodiments, a cancer cell undergoing redifferentiation gets changed into a similar or related cell type, i.e., a cell type found in the organ or tissue from which the cancer cell is derived. For example, the methods and compositions comprising acyclic retinoid and one or more aldo-keto reductase family 1 B10 (AKR1B10) inhibitors described herein are used to redifferentiate a hepatoma carcinoma cell into a hepatocyte-like cell.

“Hepatocyte-like cell,” as the term is used herein refers to a cell that has the phenotypic features of a hepatocyte, but that was derived from or generated from a non-hepatocyte precursor, such as a cancer cell following redifferentiation using any of the compositions and methods described herein, or, for example, was derived from an induced pluripotent stem cell, such as a ChiPS cell generated using any of the methods or compositions described herein. Such a “hepatocyte-like cell” has the morphological features of a hepatocyte, and can produce or express albumin at an increased level relative to a cell derived from a hepatoma cell that has not undergone dedifferentiation, for example.

Retinoids (vitamin A and its derivatives) are natural fat-soluble hormones, the biological effects of which are believed to be mediated, all or in part, by the modulation of target gene expression through two families of nuclear receptors: retinoic acid receptors (RARs) and retinoid X receptors (RXRs). Retinoids exert antitumor activity by modifying the transactivation of p21^{CIP1}, interferon receptor, and signal transduction and activator of transcription. “Acyclic retinoid” (ACR) refers to a synthetic retinoid and activates RAR and RXR. ACR is under clinical trials as a chemopreventive drug against the recurrence of HCC. Nuclear receptor RXR in HCC is highly phosphorylated through the Ras-extracellular signal-regulated kinase (ERK) pathway, inactivated, and accumulates in the line as a dominant-negative receptor. ACR inhibits the phosphorylation of RXR by inactivating the Ras-ERK pathway, recovering transactivation by retinoic acid, and induces apoptosis in human HCC cell lines (Y. Komi et al., Laboratory Investigation (2010) 90, 52-60). Accordingly, in some embodiments of the aspects described herein, a cell-growth composition or a pharmaceutical composition for redifferentiating a cancer cell comprises ACR, or a related synthetic retinoid. An exemplary acyclic retinoid for use in the compositions and methods described herein is NIK-333 [(2E,4E,6E,10E)-3,7,11,15-tetramethyl 2,4,6,10,14-hexadecapentaenoic acid, C₂₀H₃₀O₂. Other polyprenyl compounds having several linear isoprene units in the chemical structure are also contemplated for use in the methods and compositions described herein such as polyprenylcarboxylic acids having a carboxy group at the end, and conjugated polyprenylcarboxylic acids (polyprenoic acids), such as 3,7,11,15-tetramethyl-2,4,6,10,14-hexadecapentaenoic acid and esters thereof described in Japanese Patent Publication (Kokoku) No. 63-34855, and the like.

The aldo-keto reductase family 1, member B10 (aldose reductase) gene encodes a member of the aldo/keto reductase superfamily, referred to herein as “AKR1B10.” The aldo/keto reductase superfamily comprises more than 40 known enzymes and proteins. AKR1B10 member can efficiently reduce aliphatic and aromatic aldehydes, and is less active on hexoses. It is highly expressed in adrenal gland, small intestine, and colon.

Accordingly, a AKR1B10 inhibitor for use in the compositions and methods described herein refers to any agent or small molecule (e.g. a compound) that inhibits, reduces, or decreases expression or activity, such as enzymatic activity of AKR1B10. Such an refers to an agent that inhibits expression of a AKR1B10 polynucleotide or binds to, partially or totally blocks stimulation, decreases, prevents, delays activation, inactivates, desensitizes, or down regulates the activity of AKR1B10 protein. Such inhibitors include agents that, e.g., inhibit expression or stability of a polynucleotide encoding AKR1B10, or bind to, partially or totally block stimulation or any activity mediated by AKR1B10. For example, targeting of the external part of the substrate-binding site, including positions 125 and 301, where the retinoid cyclohexene ring binds, of AKR1B10, or substitutions in the cyclohexene ring of AKR1B10 can be targeted by any inhibitors against AKR1B10. Such inhibitors or antagonists include, but are not limited to, naturally occurring and synthetic ligands, antagonists, small molecules or compounds, antibodies, inhibitory nucleic acid sequence, inhibitory RNA molecules or RNA interference molecules (i.e., siRNA or antisense RNA) and the like. Such RNA interfering agents include, but are not limited to, nucleic acid molecules including RNA molecules which are homologous to the target gene or genomic sequence encoding AKR1B10, or a fragment thereof, short interfering RNA (siRNA), short hairpin or small hairpin RNA (shRNA), microRNA (miRNA) and small molecules which interfere with or inhibit expression of a target gene by RNA interference (RNAi). Inhibition of AKR1B10 expression (RNA or protein) is achieved when the expression of an AKR1B10 RNA or protein in a hepatoma cell relative to a control hepatoma cell in the absence of the inhibitor of AKRB10 is less than 80%, less than 70%, less than 60%, less than 50%, less than 40%, less than 30%, less than 20%, less than 10%, less than 5%, or when there is no detectable expression. Inhibition of AKR1B10 activity is achieved when the activity value of an AKR1B10 polypeptide relative to a control in the absence of the inhibitor of AKRB10 is less than 80%, less than 70%, less than 60%, less than 50%, less than 40%, less than 30%, less than 20%, less than 10%, less than 5%, or when there is no detectable activity.

In some embodiments, an AKR1B10 inhibitor for use in the compositions and methods described herein is tolrestat. In some embodiments, an AKR1B 10 inhibitor for use in the compositions and methods described herein is epalrestat. In some embodiments of the aspects described herein, exemplary AKR1B10 inhibitors include, but are not limited to, epalrestat or 2-[(5Z)-5-[(E)-3-cyclohexyl-2-methylprop-2-enylidene]-4-oxo-2-thioxo-3-thiazolidinyfl acetic acid (which can be obtained from commercial sources such as Aldorin (Eskayef Bangladesh Limited), Bangladesh or Aldonil (Zydus Medica), India), or a derivative thereof; 9-methyl-2,3,7-trihydroxy-6-fluorone or a derivative thereof; 5-O-Dicaffeoyl-epi-quinic acid (3,5-DCQA) or a derivative thereof; any of the siRNAs targeting AKRB10 described in “Aldo-keto Reductase Family 1 Member B10 Promotes Cell Survival by Regulating Lipid Synthesis and Eliminating Carbonyls,” The Journal of Biological Chemistry, 284, 26742-26748, herein incorporated by reference in its entirety; and any of the compounds described in “Cancer biomarker AKR1B10 and carbonyl metabolism,” Chemico-Biological Interactions Vol. 178, Issues 1-3, 2009, p. 134-137, herein incorporated by reference in its entirety.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

### EXAMPLES

### Example 1

Induced pluripotent stem (iPS) cells have been generated from human somatic cells by overexpression of defined factors, and human iPS cells can be used as research tools for drug development and regenerative medicine. However, though clinical applications of human iPS cells require the avoidance of viral transgenes, the reprogramming of human cells with only small molecules has yet to be reported. Therefore, provided herein, in some aspects, are compositions and methods for the reprogramming of human liver progenitor cells using only two small molecules. These novel compositions and methods are based, in part, on the novel discovery that using micro RNA-145 inhibitors and/or TGF-β ligand or agonists as small molecules, in human cells expressing at least some endogenous expression of OCT 3/4 and SOX2, human iPS cells can be generated using only two small molecules. These human iPS cells are termed herein as Chemicals-human induced pluripotent stem (ChiPS) cells.

In other aspects, described herein are methods to distinguish cancer cells or cells having cancerous potential in human iPS cell populations. These aspects are based, in part, on the discovery that the balance of p21 -p53 expression levels is important in order to distinguish or identify cancerous cells derived from human iPS cells. Described herein is data demonstrating that human iPS cells that carry wild-type or endogenous p53, and maintain higher expressions of p21 than p53, have significantly less risk of malignant transformations than in the cases of human iPS cells which carry mutant p53 (P<0.01). Described herein is the discovery of PRIMA-1 as a novel chemical that can be used to avoid the malignant transformations of human iPS cells which carry mutant p53. Using PRIMA-1 appropriately, unsafe human iPS cells, such as those carrying mutant p53 or having lower p53 expression, can be excluded and the best human iPS cells with high quality and safety in vitro can be chosen.

### Materials and Methods

### Cell Culture

Human Liver Biopsy Specimens. Formalin-fixed and paraffin-embedded human post-living donor transplant liver biopsy specimens were obtained in our institute. Liver biopsies from 10 living donor transplant recipients were collected at 1 week (two specimens), 6 weeks (five specimens), and 12 weeks (three specimens) post-transplant as part of a standardized protocol to rule out liver pathology following living donor transplantation. Zero specimens were collected to evaluate suspected rejection. All human tissue procedures were approved by the Institutional Review Board. Oct3/4 positive cells were observed in specimens from all timepoints post-transplantation. In specimens from 1 week, Oct3/4 positive cells were present in a contiguous streaking manner from the central vein.

### Induction of Human iPS Cells

For generation of human iPS cells, human liver progenitor cells positive for OCT 3/4 were first derived from human liver biopsy specimens. Next, the cells were seeded at a density of 5×10⁴ cells per 6-well plate in human embryonic stem (hES) cells medium. The human liver progenitor cells positive for OCT 3/4 were treated with 2′OMe-miR-145 as micro RNA-145 inhibitor (100 nmol/L; 96 hours, after that, 50 nmol/L; 72 hours) and TGF-beta ligand (100 pM; 48 hours) in the hES cells medium.

The human iPS cell (hES cells-like) colonies were then mechanically isolated and were subsequently re-plated and maintained on CF1 mouse feederlayers (Millipore) in hES cell medium.

### Western Blotting

Cells at semiconfluent state were lysed with RIPA buffer (50 mM Tris-HCl, pH 8.0, 150 mM NaCl, 1% Nonidet P-40 (NP-40), 1% sodium deoxycholate, and 0.1% SDS), supplemented with protease inhibitor cocktail (Roche). The cell lysate of MEL-1 hES cell line was purchased from Abcam. Cell lysates (20 mg) were separated by electrophoresis on 8% or 12% SDS-polyacrylamide gel and transferred to a polyvinylidine difluoride membrane (Millipore). The blot was blocked with TBST (20 mM Tris-HCl, pH 7.6, 136 mM NaCl, and 0.1% Tween-20) containing 1% skim milk and then incubated with primary antibody solution at 4° C. overnight. After washing with TBST, the membrane was incubated with a horseradish peroxidase (HRP)-conjugated secondary antibody for 1 hr at room temperature. Signals were detected with Immobilon Western chemiluminescent HRP substrate (Millipore) and LAS3000 imaging system (FUJIFILM, Japan). Antibodies used for western blotting were anti-Oct3/4 (1:600, Santa Cruz), anti-Sox2 (1:2000, Chemicon), anti-Nanog (1:200, R&D Systems), anti-Klf4 (1:200, Santa Cruz), anti-c-Myc (1:200, Santa Cruz), anti-E-cadherin (1:1000, BD Biosciences), anti-β-catenin (1:200, BD Biosciences), anti-β-actin (1:5000, Sigma), anti-mouse IgG-HRP (1:3000, Cell Signaling), anti-rabbit IgG-HRP (1:2000, Cell Signaling), and antigoat IgG-HRP (1:3000, Santa Cruz).

### Immunocytochemistry

Cultured cells were fixed with 10% formaldehyde for 10 min and blocked with 0.1% gelatin/PBS at room temperature for 1 hr. The cells were incubated overnight at 4° C. with primary antibodies against SSEA-4 (MC813-70; Chemicon), TRA-1-60 (ab16288; abcam), TRA-1-81 (ab16289; abcam), or Nanog (MAB 1997; R&D Systems), AFP (Sigma), cTNT (NeoMarkers), DESMIN (Lab Vision), GFAP (DAKO), NKX2.5 (Santa Cruz Biotechnology), PDX1 (R&D systems), SMA (Sigma), SOX17 (R&D systems), TH (Chemicon), bIII-tubulin (Covance Research Products).

For Nanog staining, cells were permeabilized with 0.1% Triton X-100/PBS before blocking. The cells were washed with PBS three times, then incubated with Alexa Fluor 488-conjugated secondary antibodies (Molecular Probes) and Hoechst 33258 (Nacalai) at room temperature for 1 hr. After further washing, fluorescence was detected with an Axiovert 200M microscope (Carl Zeiss).

qRT-PCR

Total RNA was isolated from bulk cell culture samples or from handpicked undifferentiated colonies using RNeasy columns (Qiagen) with on-column DNA digestion. cDNA was produced using oligo-dT15 priming and M-MLV reverse transcriptase (USB) according to the manufacturer's instructions at 42° C. for 1 h. About 50 ng of total RNA equivalent was typically used as template in 20 nil SYBR green PCR reactions (40 cycles of 15 s, 95° C./60 s, 60° C. on Applied Biosystems 7300 instrumentation) that additionally contained 0.375 mM of each primer and 10 nil of SYBR green PCR mix (ABI). All primers used were confirmed to amplify the predicted product at close-to-optimal efficiency without side products. Relative expression levels were calculated using the comparative Ct method, based on biological control samples and two housekeeping genes for normalization.

### In Vitro Differentiation of Human iPS Cells

For immunocytochemistry, embryoid bodies (EBs) were generated from iPS cells with the hanging-drop method in MEF-conditioned medium. After 5 days, EBs were transferred to gelatin-coated plates and subsequently cultured for another 14 days in knockout DMEM (Invitrogen) supplemented with 20% FBS, 1 mM L-glutamine, 1% non-essential amino acids, 0.1 mM b-mercaptoethanol, and penicillin/streptomycin. For qRT-PCR, iPS colonies were mechanically isolated and re-plated on Matrigelcoated plates in MEF-conditioned medium. After 2 days, medium was replaced with medium for each of the three germ layers. Endodermal differentiation: RPMI1640 medium supplemented with 2% FBS, 100 ng ml-1 activin A (R&D Systems), L-glutamine and penicillin/streptomycin for 3 weeks. For mesodermal differentiation: knockout DMEM supplemented with 100 mM ascorbic acid (Sigma), 20% FBS, 1 mM L-glutamine, 1% non-essential amino acids, 0.1 mM b-mercaptoethanol and penicillin/streptomycin for 3 weeks. For ectodermal differentiation: the cells were maintained in N2B27 medium for 7 days and the medium replaced with N2 medium supplemented with 10 ng ml-1 bFGF2 (peprotech), 100 ng ml-1 Sonic Hedgehog (R&D Systems), 10 ng ml-1 PDFG (R&D Systems), L-glutamine and penicillin/streptomycin for 2 weeks. The medium was changed every other day. Furthermore, human normal hepatocytes were generated from ChiPS cells in vitro, according to reported methods (11).

### Bisulfite Sequencing

Genomic DNA (1 mg) was treated with a CpGenome DNA modification kit (Chemicon) according to the manufacturer's recommendations. Treated DNA was purified with a QIA quick column (QIAGEN). The promoter regions of the human Oct3/4 and Nanog genes were amplified by PCR. The PCR products were subcloned into pCR2.1-TOPO. Ten clones of each sample were verified by sequencing with the M13 universal primer. Primer sequences used for PCR amplification were provided in a previous report (3).

### Karyotyping and DNA Fingerprinting Analysis

Chromosomal G-band analyses and DNA fingerprinting analysis were performed in our laboratories.

### Teratoma Formation

Teratoma formation was performed as previously described (3).

### Primers for RT-PCR Gene Primer Sequence

Primers for RT-PCR gene primer sequence were selected and used as previously described (3). As for β-catenin, the primers for RT-PCR gene primer sequence selected and used as previously described (12).

### The Evaluation of Cancerous Risk for the Human iPS Cell Lines

The various human iPS cell lines (No. 1-No. 4) were inoculated intramuscularly into immunodeficient mice (Rag2^{-/-} Il2rg^{-/-}). Then, microvessel density (MVD) was compared within teratomas in mice among each group. Furthermore, for angiogenesis in teratomas from each human iPS cell lines, microvessel density (MVD) per high-powered field (h.p.f.) of teratomas was quantified by human-specific anti-CD31 immunofluorescence. n=3-6; All values were mean±s.e.m.

### Results

The Generation of Human Induced Pluripotent Stem (iPS) Cells

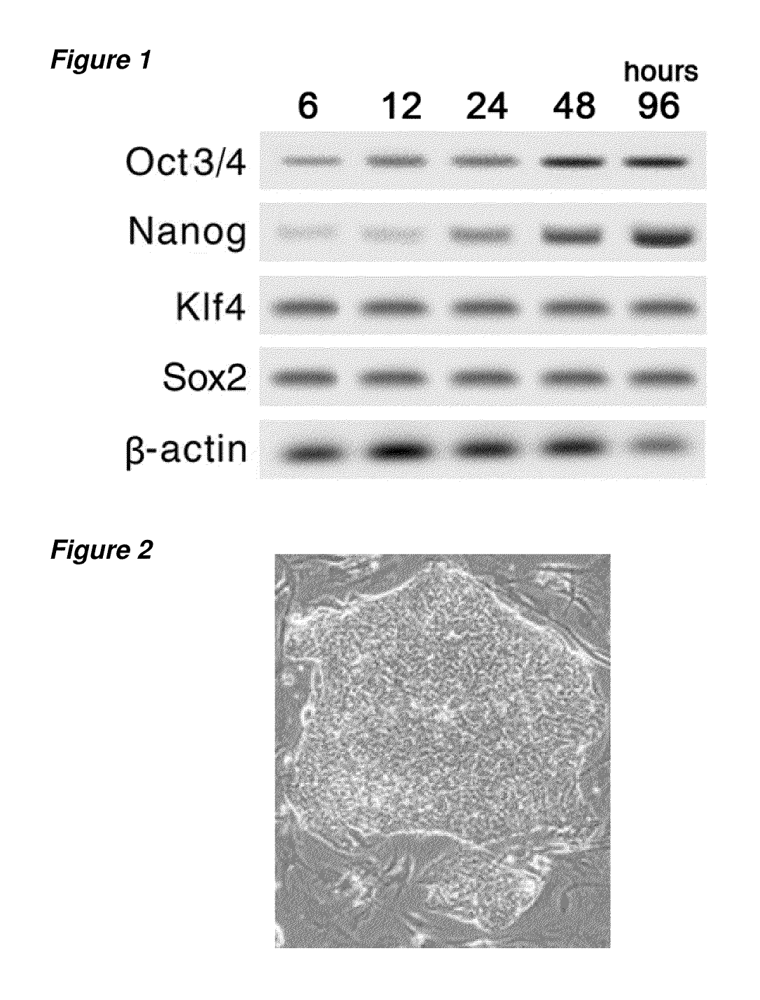

As shown in FIG. 1, administration of small molecules resulted in the reactivation of OCT3/4, SOX2, KLF4 and NANOG. However, by stopping administration, the reactivation of the expression of each pluripotency-associated gene was reversed or stopped, i.e., the genes were silenced. Around day 14 following the administration of the small molecules, human hES cell-like colonies were observed, as shown in FIG. 2.

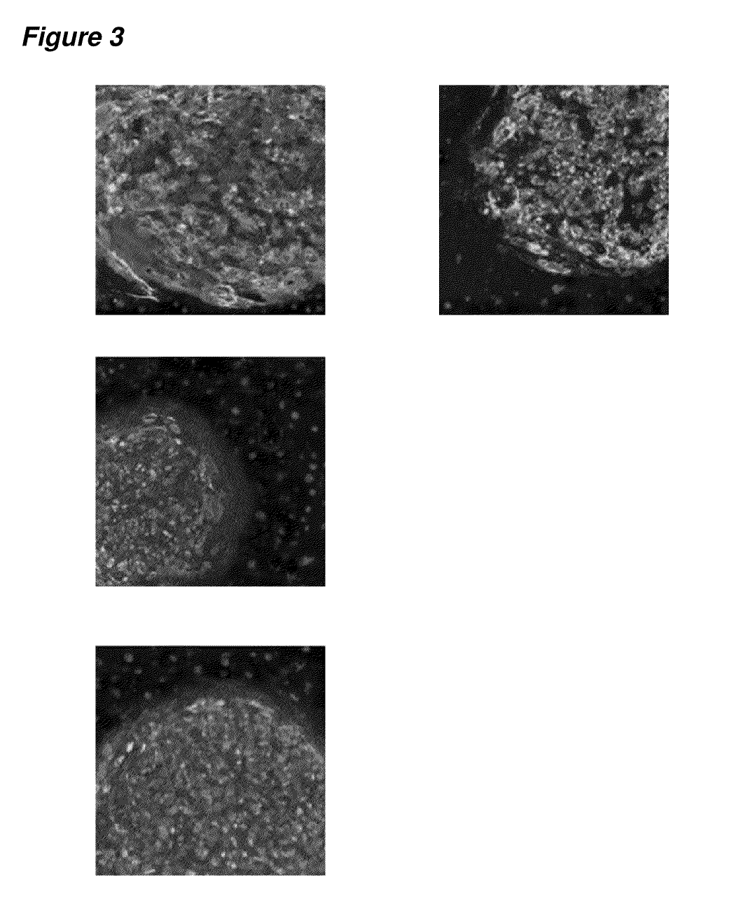

As shown in FIG. 3, the cells that were generated using the small molecule compounds described herein expressed hES cell-specific surface antigens, including SSEA-4, tumor-related antigen (TRA)-1-60, TRA-1-81 and Nanog protein.

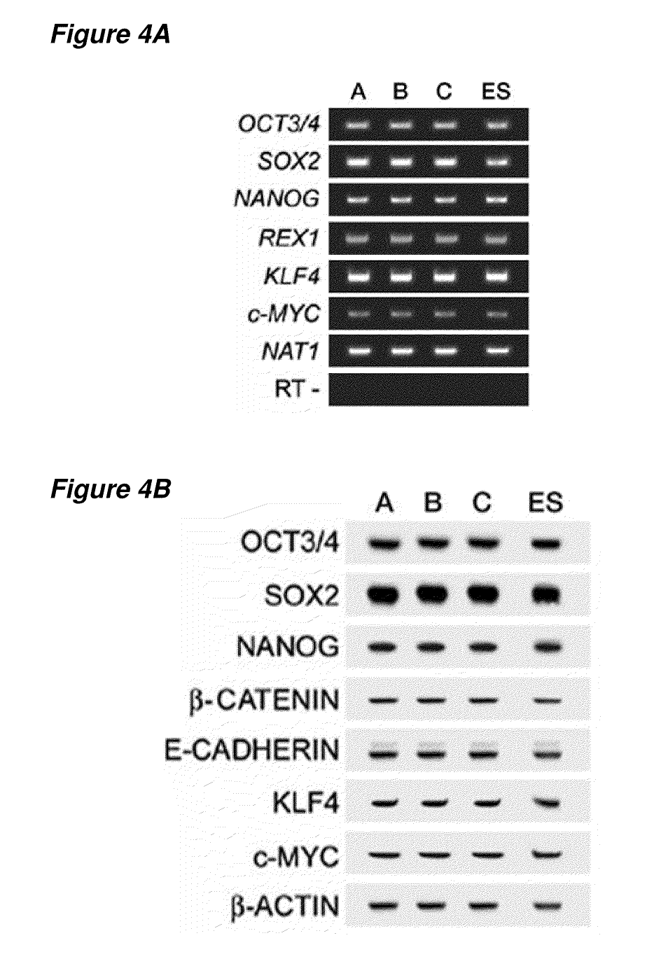

Furthermore, reverse transcription polymerase chain reaction (RT-PCR) showed that the cells that were generated using the small molecule compounds described herein expressed many undifferentiated ES cell-marker genes such as OCT3/4, SOX2, and NANOG, etc, at levels equivalent to the hES cell line H9 (FIG. 4A). Moreover, Western blotting analysis of protein levels of OCT3/4, SOX2, and NANOG, were found to be similar between the cells that were generated using the small molecule compounds described herein and hES cells (FIG. 4B).

Bisulfite genomic sequencing analyses evaluating the methylation status of cytosine guanine dinucleotides (CpG) in the promoter regions of pluripotent-associated genes, such as OCT3/4, and NANOG, revealed that they were highly unmethylated (FIG. 5), thus further demonstrating that the cells that were generated using the small molecule compounds described herein were highly similar to hES cells. Accordingly, the cells that were generated using the small molecule compounds described herein were termed as “chemical induced pluripotent stem cells” (“ChiPS cells”).

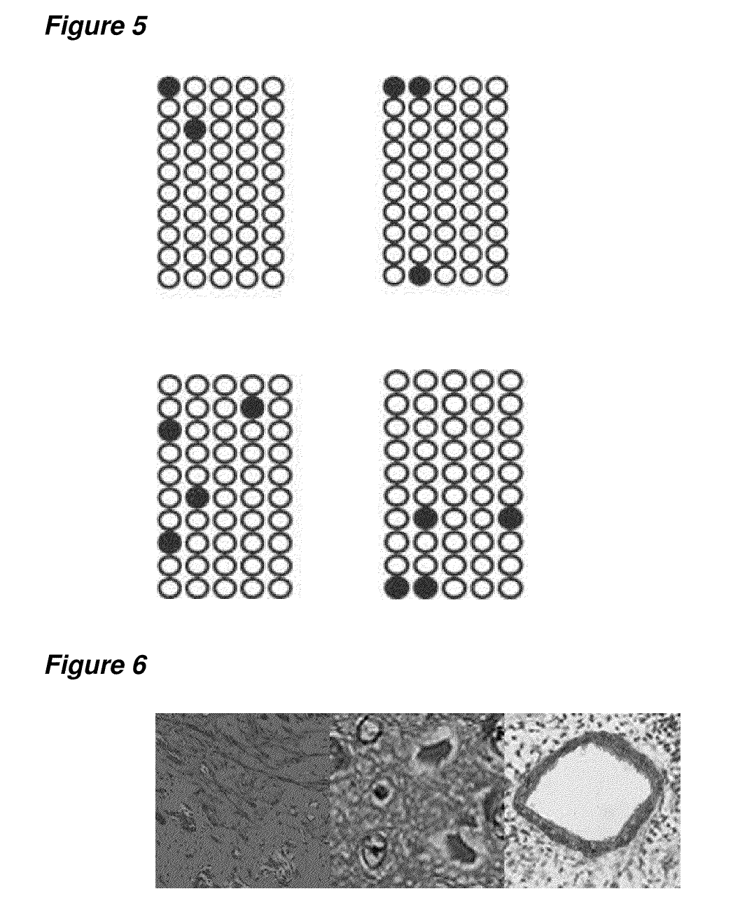

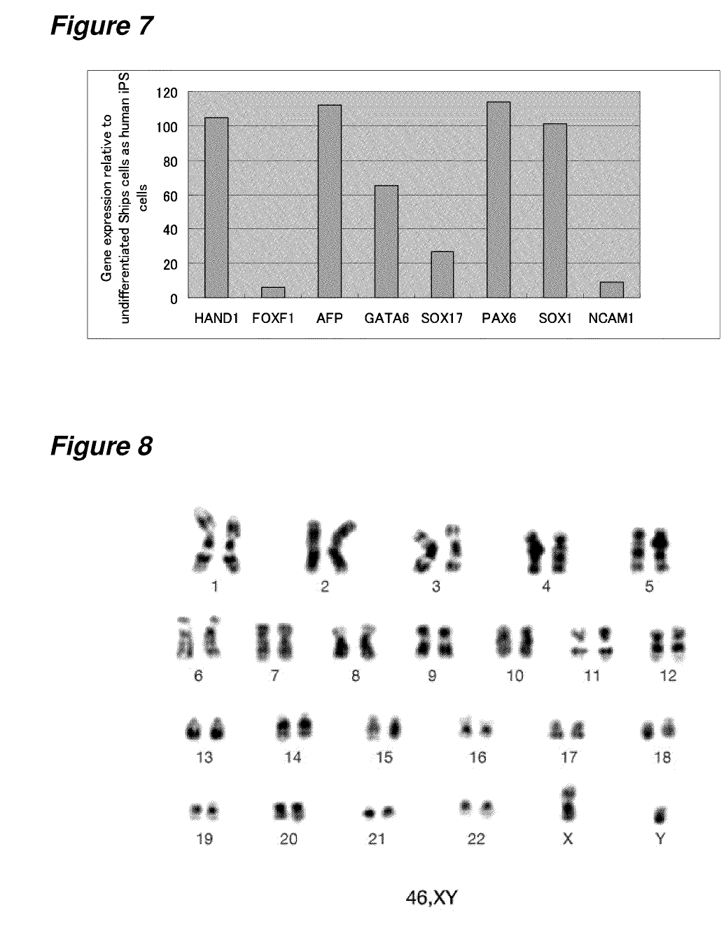

Further, in order to test pluripotency in vivo, ChiPS cell lines were transplanted subcutaneously as human iPS cell lines into the dorsal flanks of immunodeficient (SCID) mice. Nine weeks after injection, tumor formation was observed. Histological examination showed that the tumor contained neural tissues (ectoderm), striated muscle (mesoderm), and gut-like epithelial tissues (endoderm) (FIG. 6), demonstrating that the ChiPS cells were pluripotent. Moreover, these ChiPS cells could differentiate into cell types of the three germ layers in vitro (FIG. 7). For example, we could human generate normal hepatocytes from ChiPS cells in vitro using reported methods (11).

Further, chromosomal G-band analyses showed that human ChiPS cells had a normal karyotype of 46 XY (FIG. 8). In addition, DNA fingerprinting analysis confirmed that human iPS cells were liver progenitor cells in origin. Thus, human ChiPS cell clones were derived from liver progenitor cells and were not a result of cross-contamination. Therefore, described herein is generation of human iPS (ChiPS) cells from human somatic cells using only small molecule compounds.

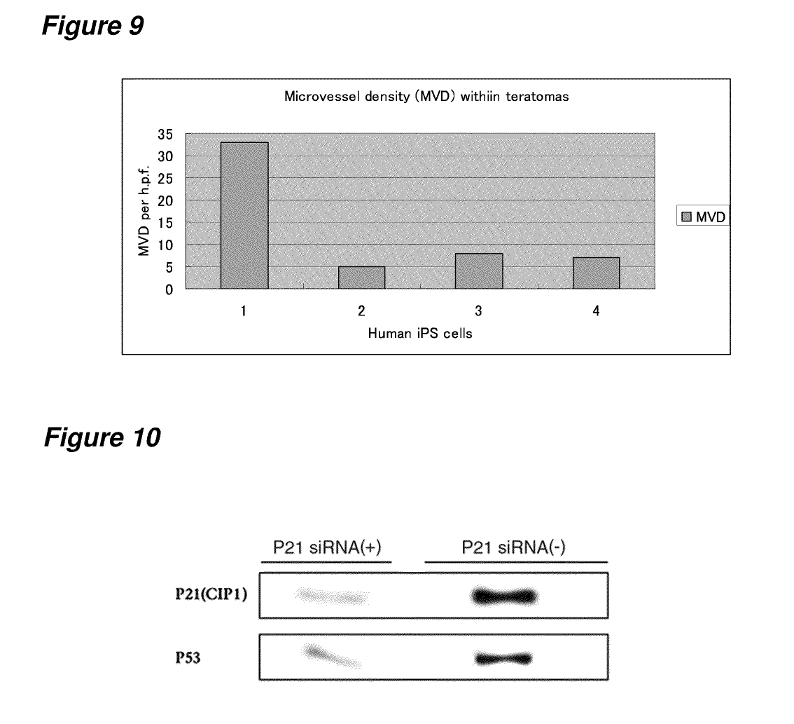

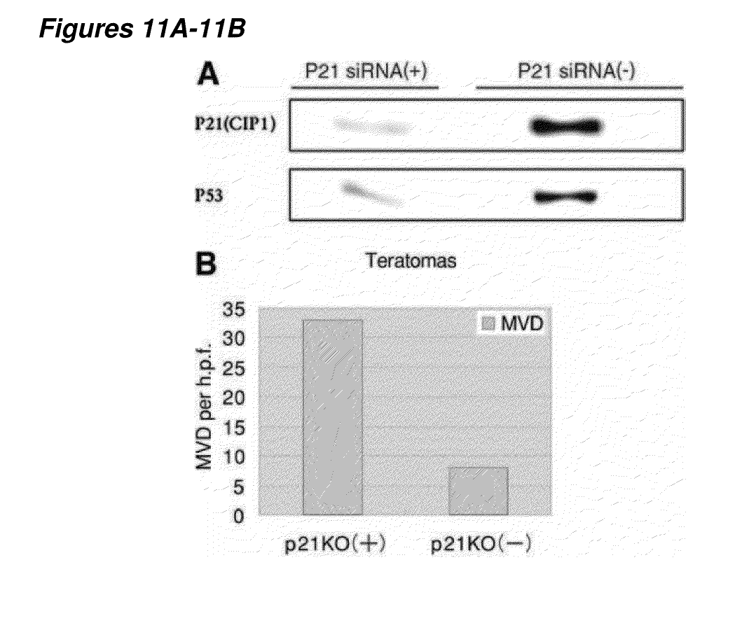

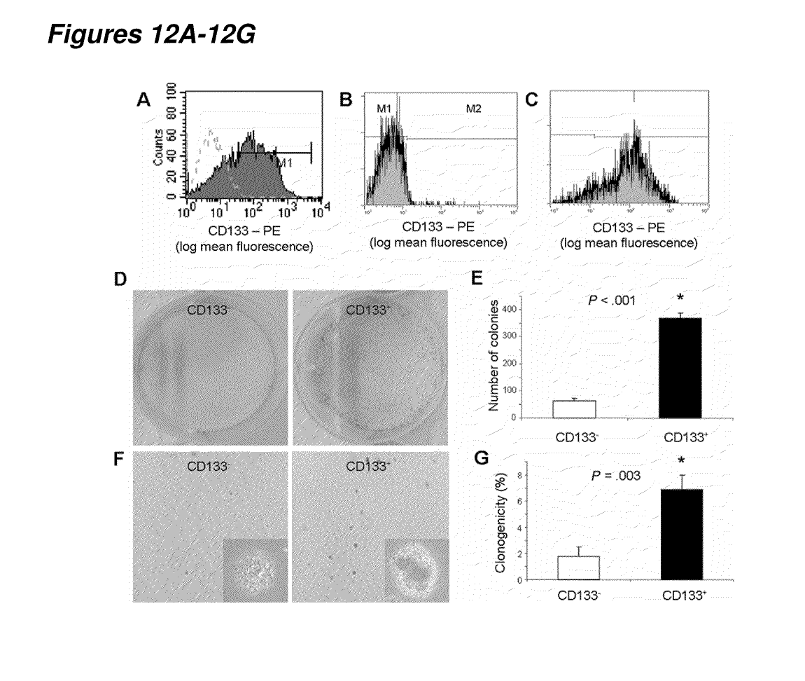

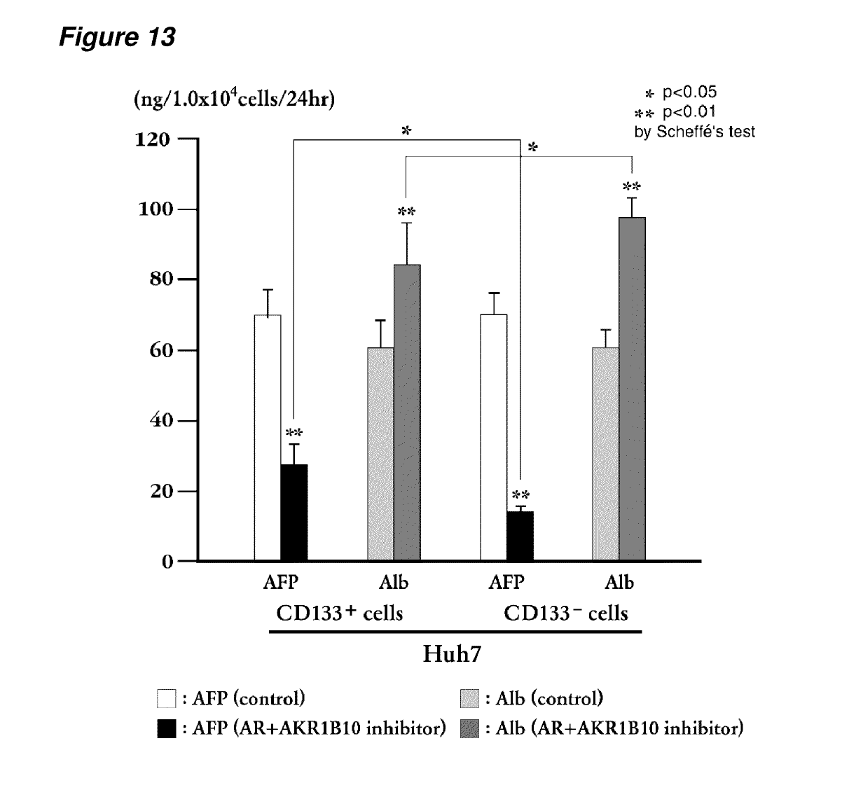

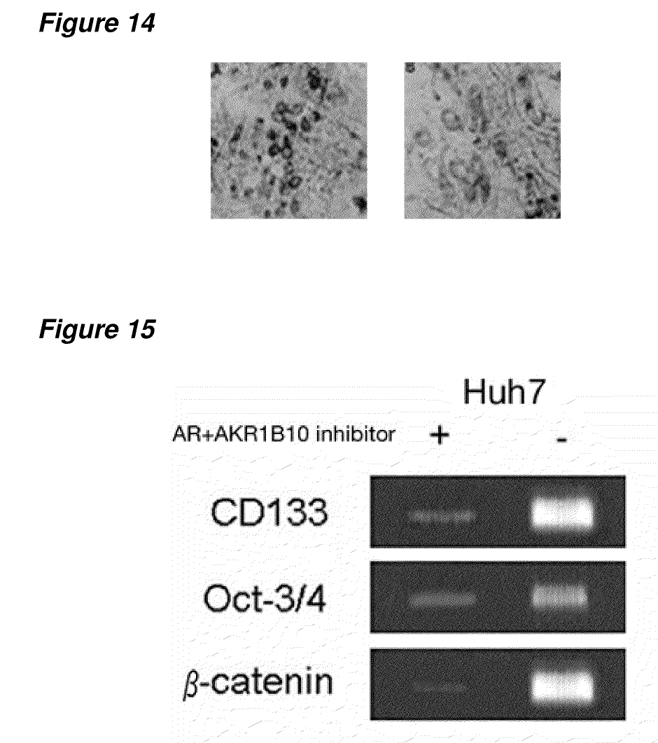

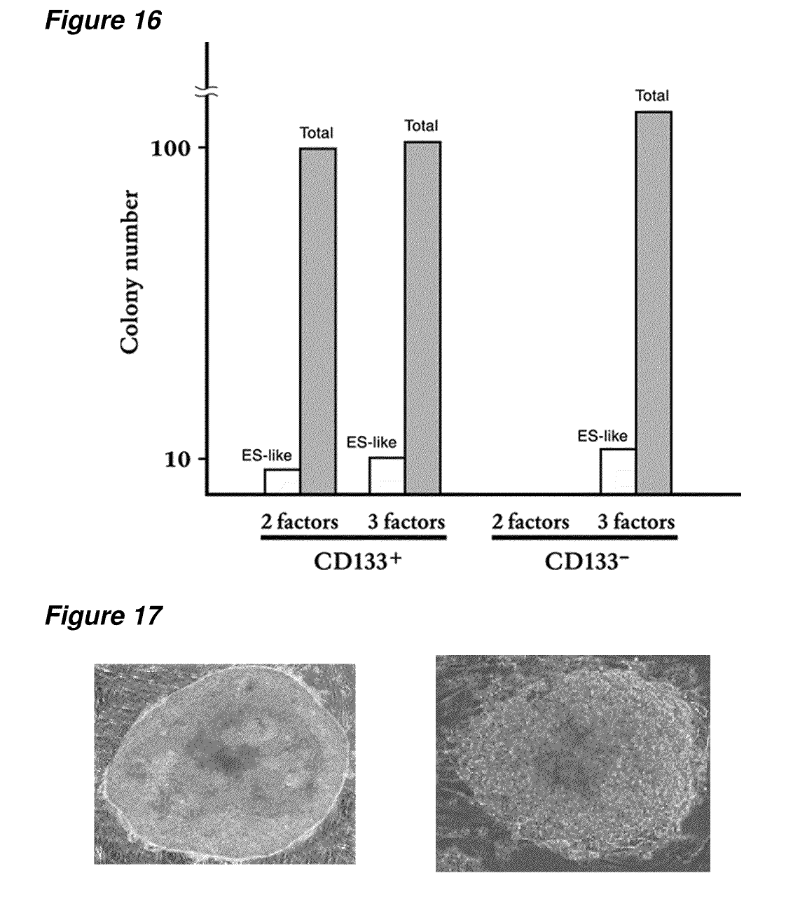

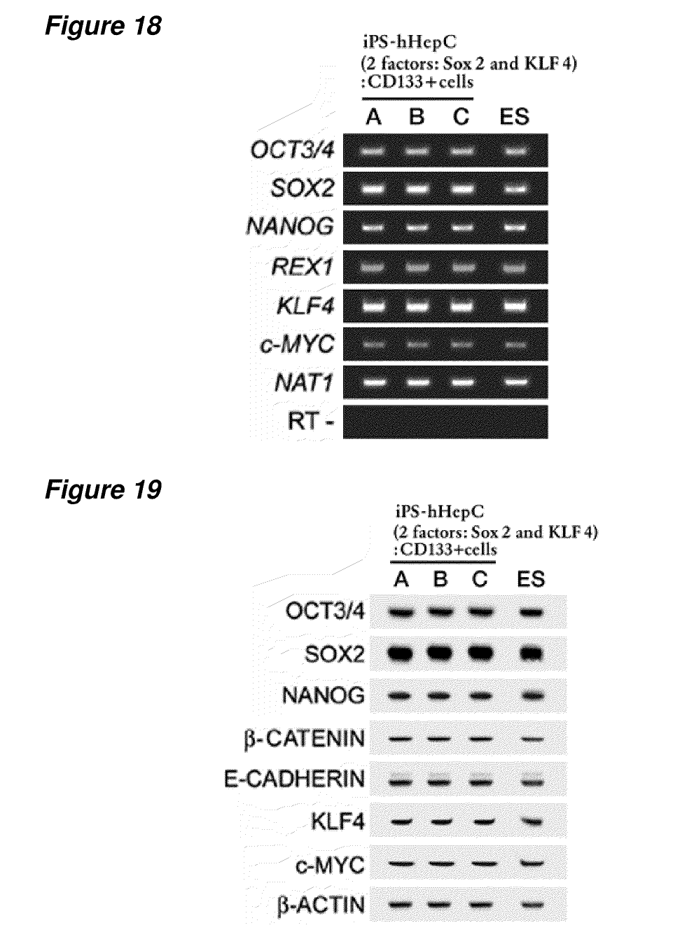

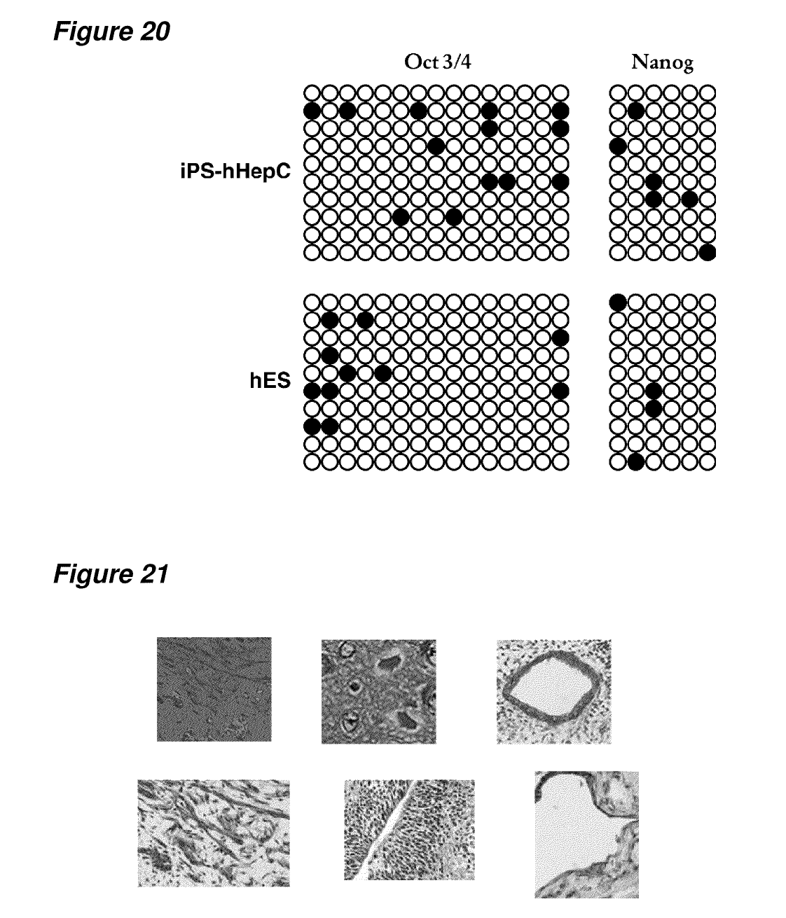

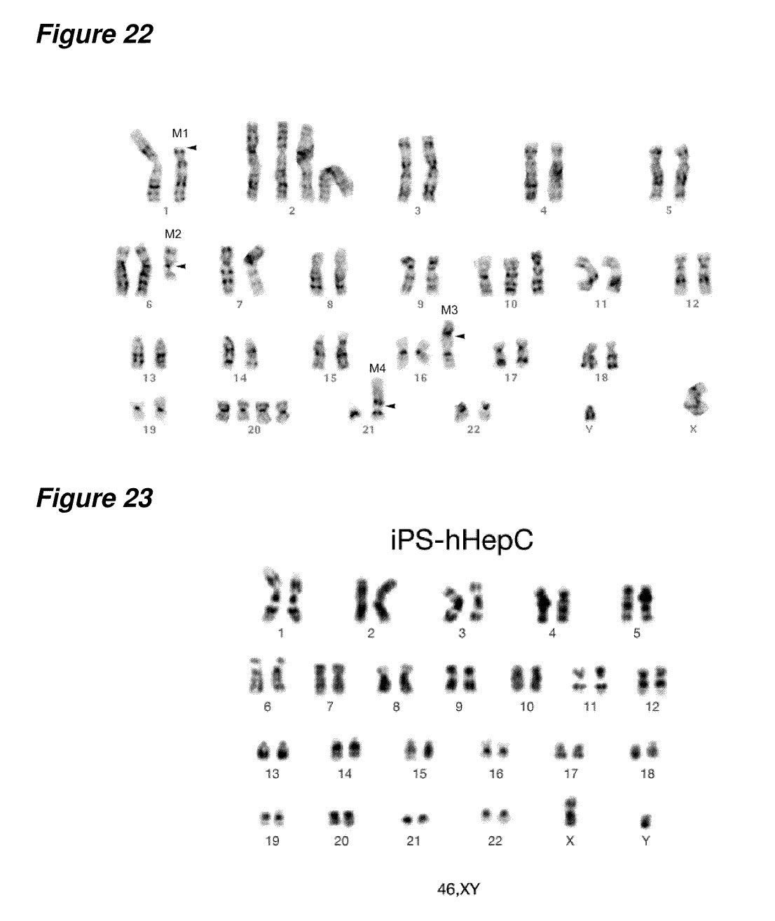

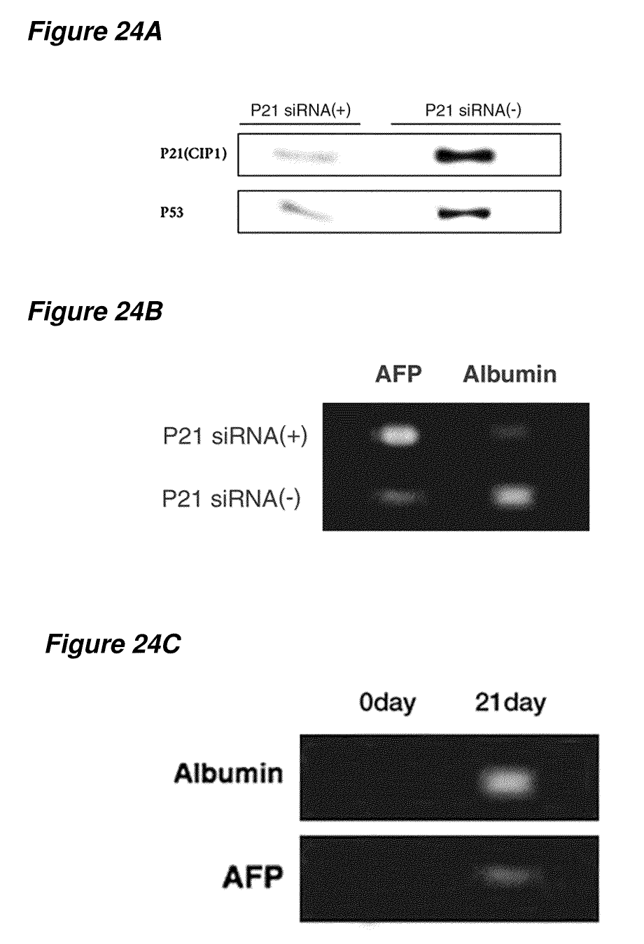

By using microvessel density analysis (MVD) within SCID mice that were transplanted with ChiPS cell lines, risk evaluation of malignant transformations were performed for human iPS cell lines established by various methods (3) (4) (5) (6), including the results described herein (FIG. 9). These data demonstrate that human iPS cells that carry mutant p53 have greater risk for malignancy by using the evaluation of MVD (FIG. 9). Then, p53-175N, p53-175S and p53-175D were identified as p53-mutational spots in human iPS cells which carry mutant p53, with codon 175 in human p53 being known as one mutational spot in human cancer (7).

Furthermore, human iPS cells that carried mutant p53 were either partially reprogrammed and/or were iPS cells established using viral transgenes. In contrast, all human iPS cell lines established by small molecules in the present study carried wild p53, and p53 expressions within the cells were controlled comparing with p21 expressions.

Next, human iPS cells that carry mutant p53 were selected and treated with PRIMA-1 (20 μM). As a result, MVD in human iPS cells carrying mutant p53 became equivalent to that of human iPS cell lines established by small molecules (FIG. 8). Furthermore, though it was previously found the induction of p21 is necessary to avoid malignant transformations of various human iPS cells (8), PRIMA-1 (9) induced the expression of p53 target genes p21 within human iPS cells which carry mutant p53, and induction of p53-dependent apoptosis in human iPS cells that have mutant p53 was found. In contrast, induction of p53-dependent apoptosis in human iPS cells having wild-types p53 treated with PRIMA-1 was not observed.

### Discussion

Described herein are methods and compositions for generating human iPS cells from human liver progenitor cells using only small molecules, termed herein as ChiPS cells. The human iPS cells were similar to hES cells in morphology, proliferation, surface antigens, gene expression, and epigenetic status of pluripotent cell-specific genes. Furthermore, these cells could differentiate into cell types of the three germ layers in vitro and into teratomas. The reprogramming efficiency of generating human iPS cells ranged from 0.03-0.05%. Therefore, the efficiency was improved over previous reports (3) (4) (5) (6).

Next, it was discovered that MVD was the least when ChiPS cells were used as human iPS cells. Without wishing to be bound or limited by theory, this observation demonstrates that a profile that ChiPS cells have least risk for malignant transformations compared to other human iPS cells and lines. On the other hand, though induction of p53-dependent apoptosis was not found in the human iPS cells that carry wild p53 treated with PRIMA-1 (9) (20 μM), using induction of p53-dependent apoptosis of human iPS cells that carry mutant p53 by PRIMA-1 (9) (20 μM), unsafe human iPS cells that carry mutant p53 can be excluded, and human iPS cells with high quality and safety in vitro can be chosen, regardless of the method or methods used to generate human iPS cells (3)(4)(5)(6). Therefore, PRIMA-1 (20 μM) is an important small molecule for use in clinical application of human iPS cells.

In conclusion, described herein are compositions and methods for generating and using ChiPS cells using only small molecules. Furthermore, described herein are methods for using p21 -p53 expression levels in order to distinguish or identify cancer cells or cells with potential for developing into cancerous cells in populations of human iPS cells. It was found that human iPS cells that carry wild-type p53 maintained higher expressions of p21 than p53, and that the risk of malignant transformations of human iPS cells was significantly less than those human iPS cells that have mutant p53 (FIG. 8, P<0.01). Further, it was found that while human iPS cells carrying mutant p53 have high risk for malignant transformations (FIG. 8), PRIMA-1 was identified as a novel chemical to avoid the malignant transformations of human iPS cell carrying mutant p53 generated using any method (3) (4) (5) (6).

### Example 2

Described herein are novel methods and compositions for generating hepatocyte-like cells from human CD133⁺ hepatoma cells and the induction of iPS cells from the hepatocyte-like cells (iPS-hHepC) using only Sox2, Klf4 and chemicals.

As demonstrated herein, iPS-hHepC cells expressed hES cell-specific surface antigens, and RT-PCR showed that iPS-hHepC expressed many undifferentiated ES cell-marker genes at levels equivalent to the hES cell line H9. Further, Western blotting analyses shown here demonstrate that protein levels of Oct3/4, Sox2, and Nanog, for example, were similar between iPS-hHepC and endogenous hES cells. In addition, bisulfite genomic sequencing analyses evaluating the methylation status of cytosine guanine dinucleotides in the promoter regions of pluripotent-associated genes revealed that they were highly unmethylated in iPS-hHepC cells.

Transplantation of iPS-hHepC into SCID mice revealed that nine weeks after injection, tumor formation was observed. Histological examination showed that the tumor contained neural tissues, striated muscle, and gut-like epithelial tissues, demonstrating that the iPS-hHepC were pluripotent. Chromosomal G-band analyses showed that iPS-hHepC had a normal karyotype of 46 XY. In addition, DNA fingerprinting analysis confirmed that iPS-hHepC were iHep origin. The reprogramming efficiency of generating iPS-hHepC ranged from 0.014% to 0.022%.

Further analyses of p21 and p53 expression revealed that expression of p53 was controlled in comparison with p21 in iPS-hHepC cells. It was found that knockdown of p21 in iPS-hHepC cells during a differentiation induction process to hepatocyte cells caused transformation to human hepatoma-like cells, similar to those cells from which iPS-hHepC cells were originally derived. In contrast, in the absence of knockdown of p21, the iPS-hHepC cells could differentiate to normal human hepatocyte-like cells as characterized by expression of albumin in 21 days. Accordingly, described herein are methods for assessing the risk of tumor transformation by comparison of expression of p21 and p53, and methods for preventing cancerous transformation of t by maintaining higher expression of p21 than p53. Further, an important role of Klf4n in order to prevent tumor transformation of human iPS cells is described herein.

### REFERENCES

1. Xu N, MicroRNA-145 regulates Oct 4, Sox2, and Klf4 and represses pluripotency in human embryonic stem cells. Cell 2009; 137: 647-658.
2. Ikushima H, Todo T, Ino Y, et al. Autocrine TGF-beta signaling maintains tumorigenicity of glioma-initiating cells through Sry-related HMG-box factors. Cell Stem Cell 2009; 5: 504-514.
3. Takahashi K, Tanabe K, Ohnuki M, et al. Induction of pluripotent stem cells from adult human fibroblasts by defined factors. Cell 2007; 131: 861-72.
4. Park I H, Zhao R, West J A, Yabuuchi A, Huo H, et al. Reprogramming of human somatic cells to pluripotency with defined factors. Nature 2008; 451: 141-46.
5. Nakagawa M, Koyanagi M, Tanabe K, Takahashi K, Ichisaka T, et al. (2008) Generation of induced pluripotent stem cells without Myc from mouse and human fibroblasts. Nature Biotechnology 2008; 26: 101-6.
6. Yu J, Hu K, Smuga-Otto K, et al. Human induced pluripotent stem cells free of vector and transgenes sequences. Science 2009; 324:797-801.
7. Cho. Y, Gorina S, Jeffrey P D, et.al. Crystal structure pf a p53 tumor suppressor-DNA complex:] understanding tumorigenic mutations. Science 1994; 265:346-355.
8. Moriguchi H, Chung R T, Sato C. An indicator in order to evaluate the risk for cancerous transformations of human induced pluripotent stem cells. Hepatology 2010; 51: 1085-1086.
9. Bykov V, Issaeva N, Shilov A, et al. Restoration of the tumor suppressor function to mutant p53 by a low molecular weight compound. Nature Medicine 2002; 8: 282-288.
10. Mihara M, Nakagawa T, Noguchi S, et al. MRI, Magnetic resonance influenced, organ freezing method under magnetic field. ACSC 2009; 1: 34-37.
11. Sullivan G J, Hay D C, Park I H, et al. Generation of functional human hepatic endoderm from human induced pluripotent stem cells. Hepatology 2010; 51: 329-335.
12. Ma S, Lee T K, Zheng B J, Chan K W, Guan X Y. CD133+HCC cancer stem cells confer chemoresistance by preferential expression of the Akt/PKB survival pathway. Oncogene 2008; 27: 1749-58.

We claim:

1. A method of generating a chemically induced pluripotent stem (ChiPS) cell from a human somatic cell, the method comprising contacting a human somatic cell with an inhibitor of microRNA-145 and an activator of TGF-β signaling, wherein a chemically induced pluripotent stem cell is generated.

2. The method of claim 1, wherein the human somatic cell has endogenous expression of OCT3/4.

3. The method of claim 1, wherein the human somatic cell has endogenous expression of SOX2.

4. The method claim 1, wherein the inhibitor of microRNA-145 is a small molecule inhibitor of microRNA-145 or a nucleic acid inhibitor of microRNA-145.

5. The method of claim 4, wherein the nucleic acid inhibitor of microRNA-145 is a anti-microRNA-145 LNA oligonucleotide or a 2′OMe-microRNA-145.

6. The method of claim 1, wherein the activator of TGF-β signaling is a TGF-β polypeptide or an active fragment thereof, a fusion protein comprising a TGF-β polypeptide or an active fragment thereof, an agonist antibody to a TGF-β receptor, a small molecule agonist of a TGF-β receptor, or a TGF-β production stimulator.

7. The method of claim 1, further comprising the step of measuring an expression of p21 and measuring an expression of p53 in the generated chemically induced pluripotent stem cell, wherein an increased expression of p21 relative to the expression of p53 in the induced pluripotent stem cell is indicative of a decreased risk of cancerous transformation.

8. A cell growth composition comprising an inhibitor of microRNA-145 and an activator of TGF-β signaling.

9. The composition of claim 8, further comprising a human somatic cell in admixture with the inhibitor of microRNA-145 and the activator of TGF-β signaling.

10. The composition of claim 8, wherein the human somatic cell has endogenous expression of OCT3/4.

11. The composition of claim 8, wherein the human somatic cell has endogenous expression of SOX2.

12. The composition of claim 8, wherein the inhibitor of microRNA-145 is a small molecule inhibitor of microRNA-145 or a nucleic acid inhibitor of microRNA-145.

13. The composition of claim 12, wherein the nucleic acid inhibitor of microRNA-145 is a anti-microRNA-145 LNA oligonucleotide or a 2′OMe-microRNA-145.

14. The composition of claim 8, wherein the activator of TGF-β signaling is a TGF-β polypeptide or an active fragment thereof, a fusion protein comprising a TGF-β polypeptide or an active fragment thereof, an agonist antibody to a TGF-β receptor, a small molecule agonist of a TGF-β receptor, or a TGF-β production stimulator.

15. A method of assessing the risk of cancerous transformation of an induced pluripotent stem (iPS) cell or population of induced pluripotent stem cells, the method comprising measuring an expression of p21 and measuring an expression of p53 in an induced pluripotent stem cell or population of induced pluripotent stem cells, wherein an increased expression of p21 relative to the expression of p53 in the induced pluripotent stem cell is indicative of a decreased risk of cancerous transformation.

16. A method of decreasing the risk of cancerous transformation of an induced pluripotent stem (iPS) cell, the method comprising contacting an induced pluripotent stem cell with one or more agents that induce p21 expression in the induced pluripotent stem cell, wherein the induction of p21 expression in the induced pluripotent stem cell decreases the risk of cancerous transformation.

17. The method of claim 16, wherein the agent that induces p21 expression is PRIMA-1 (2,2-Bis(hydroxymethyl)-1-azabicyclo[2.2.2]octan-3-one).
